SEQUENCE LISTING (1 sequence)

SEQ ID NO: 1 - protein, 12 aa, Homo sapiens
RKRLQVQLSIRT

(19) United States
(12) Patent Application Publication
(10) Pub. No.: US 2026/0199514 A1
(43) Pub. Date: Jul. 16, 2026

(54) PRODRUG STRATEGY THAT ENHANCES EFFICACY AND LOWERS SYSTEMIC TOXICITY OF MERTANSINE

(71) Applicant: UNIVERSITY OF NOTRE DAME DU LAC, South Bend, IN (US)

(72) Inventors: Zihni Basar BILGICER, Granger, IN (US); Sabrina KHAN, South Bend, IN (US); Franklin MEJIA, South Bend, IN (US)

(73) Assignee: UNIVERSITY OF NOTRE DAME DU LAC, South Bend, IN (US)

(21) Appl. No.: 19/129,916

(22) PCT Filed: Nov. 17, 2023
(86) PCT No.: PCT/US2023/080373

§ 371 Date: May 14, 2025

Related U.S. Application Data
(60) Provisional application No. 63/384,352, filed on Nov. 18, 2022.

(51) Int. Cl. A61K 47/69; A61K 47/54; A61K 47/62; A61P 35/00

(52) U.S. Cl. CPC A61K 47/6911; A61K 47/544; A61K 47/62; A61P 35/00

(57) ABSTRACT

A nanoparticle and methods of using the same wherein the nanoparticle includes a prodrug comprising a targeting moiety-lipid conjugate, a polyethylene glycol-lipid conjugate, a sterol, a bulk lipid, and a drug-lipid conjugate, wherein a chemical linker group is positioned between the drug moiety and the lipid moiety of the drug-lipid conjugate. The nanoparticle can be used to treat cancer, for example, multiple myeloma.

### RELATED APPLICATIONS

The present application claims priority under 35 U.S.C. § 119(e) to U.S. Provisional Patent Application No. 63/384,352 filed Nov. 18, 2022, the entire disclose of which is hereby incorporated by reference in its entirety.

### SEQUENCE LISTING

The instant application contains a Sequence Listing which has been submitted electronically in ST.26 format and is hereby incorporated by reference in its entirety. The ST.26 copy, created on Nov. 17, 2023, is named 501-103W01_SL, and is 1,817 bytes in size.

### BACKGROUND

Chemotherapy is still one of the most commonly employed and effective strategies for treating advanced and metastatic tumors. Nevertheless, conventional methods of chemotherapeutic delivery have a narrow therapeutic window and typically result in high systemic toxicity, leading to poor efficacy with serious adverse effects in patients. For several decades, nanoparticles, especially liposomal based versions, have been dominant in clinical investigations ranging from cancer treatment, vaccines, gene therapies, contrast agents and other applications. To date, more than 30 nanoparticles have been approved in various clinical applications, and at least 8 of these formulations are liposomal nanoparticles for treating several cancers at various stages. All FDA approved liposomal nanoparticle formulations to date have been designed without utilizing any active targeting moieties. Most important characteristics of these nanoparticles are their ability to home into tumor microenvironment due to tumor vasculature and defective lymphatic drainage (also known as enhanced permeation and retention (EPR) effect), reduce systemic toxicity and limit off-target effects. Although, the consensus in the field is that targeted nanomedicine would provide significant improvement in cancer treatments, so far, their translation to the clinic has been poor. This has been due to reasons relating to insufficient efficacy, and problems relating to scaling up and other manufacturing concerns, animal models that fall short of emulating the clinical human condition, as well as issues relating to reproducibility of preclinical studies during trials.

Targeting of specific cells takes advantage of certain receptors being overexpressed on cancer tissue over healthy cells. Therefore, traditional antibody targeted therapies (or similars such as Fab, scFv, etc.) have reports of exhibiting poor selectivity and off-target related toxicity. This is typically due to the drug carrier binding to the target receptor whether it is presented on diseased or healthy tissue. The antibody's high affinity results in a slow dissociation, which in turn increases the residence time and likelihood of delivery of the chemotherapeutic indiscriminately to the tissue.

Accordingly, there is a need for new compounds and methods for treating cancers that include the advantages of lower antigenicity, decreased opsonization, and increased stability of the delivery system, as well as facile and precise synthesis, and lower cost. The present disclosure satisfies these needs.

### SUMMARY OF THE INVENTION

In the present disclosure, we investigated formulating DM1 (also known as Mertansine) as a part of a targeted nanomedicine to be used as an anti-cancer therapeutic. Despite its high potency for treating cancer, DM1 is being used in a very limited clinical capacity due to its devastating side-effects, including gastrointestinal reactions, liver damage, and peripheral neurotoxicity. Currently, DM1 applications in the clinic are limited to an antibody-drug conjugate (ADC) for late-stage breast cancer and HER2-positive early breast cancer treatments since its approval by the FDA in 2013 and 2019 respectively. We hypothesize that developing a targeted nanoparticle formulation would make DM1 a strong contender in the arsenal for the fight against various types of cancers, potentially introducing it as a treatment option for a broader spectrum of cancers going beyond just breast cancer. Here, we evaluated several prodrug versions of DM1 for its use in the treatment of multiple myeloma (MM), which is the second most common hematologic malignancy in the United States.

Described herein is the development of peptide-targeted-liposomal-DM1 prodrug (TNP[Prodrug]) formulations to achieve selective drug delivery and release to treat cancer, such as MM, with reduced side effects. The prodrugs are designed to have a hydrophobic lipid tail to facilitate their anchoring to the lipid bilayer of the liposomes for their guaranteed incorporation into the nanoparticles. We designed and synthesized four different DM1-Prodrugs as drug-lipid conjugates using linker moieties with different chemistries. As the targeting element, we utilized a CD138 peptide (CD138pep). Next, liposomal formulations prepared using CD138pep and various DM1-Prodrugs were evaluated using in vitro and in vivo mouse cancer models to rate efficacy. Using DM1-Prodrugs with different linker chemistries in our approach has enabled us to identify the most efficacious TNP[Prodrug-4] formulation in vitro and achieve formidable inhibition of tumor growth in vivo with negligible systemic toxicity.

Accordingly, the disclosure provides for a nanoparticle comprising a prodrug comprising a drug-lipid conjugate, wherein a chemical linker group is positioned between a drug and a lipid of the drug-lipid conjugate; a targeting moiety-lipid conjugate; a polyethylene glycol-lipid conjugate; a sterol, such as cholesterol; and a bulk lipid.

In some embodiments, the drug of the drug-lipid conjugate comprises mertansine (DM1):

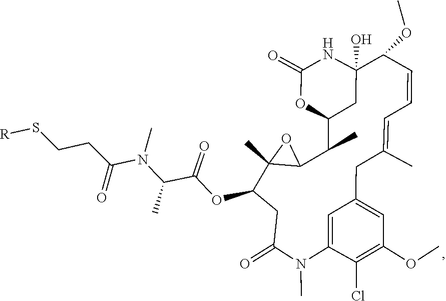

wherein R is the chemical linker group that links the drug to the lipid of the drug-lipid conjugate.

In some embodiments, the chemical linker group comprises one of an amide linker, an ester linker, a disulfide linker, and a phosphodiester linker. In various embodiments, a) the lipid of the drug-lipid conjugate and the amide linker comprise moiety (1):

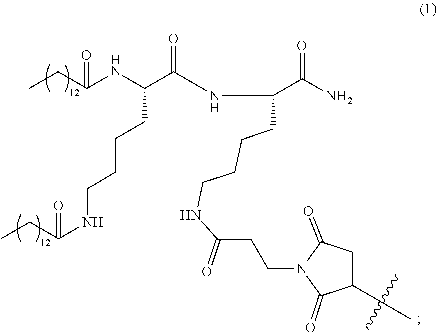

b) the lipid of the drug-lipid conjugate and the ester linker comprise moiety (2):

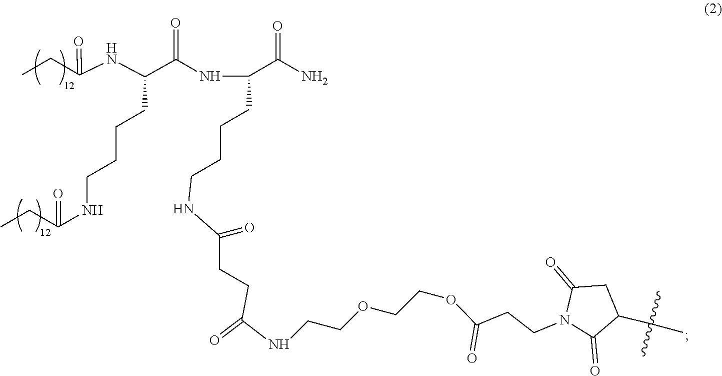

c) the lipid of the drug-lipid conjugate and the disulfide linker comprise moiety (3):

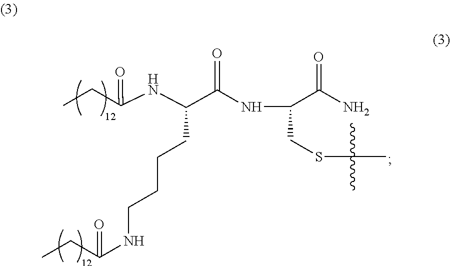

d) the lipid of the drug-lipid conjugate and the phosphodiester linker comprise moiety (4):

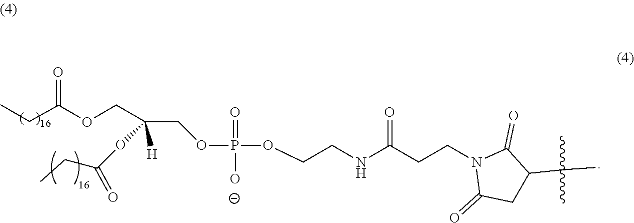

In some embodiments, the prodrug comprises one or more of:

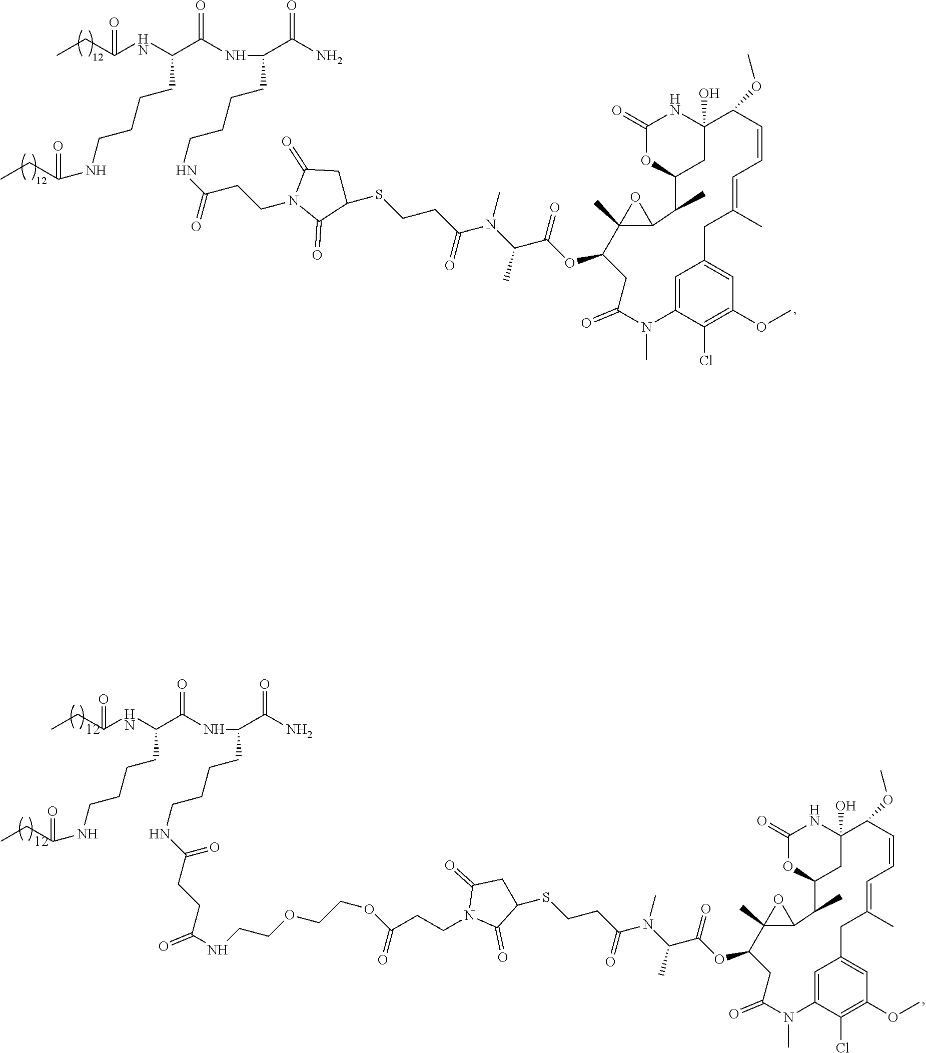

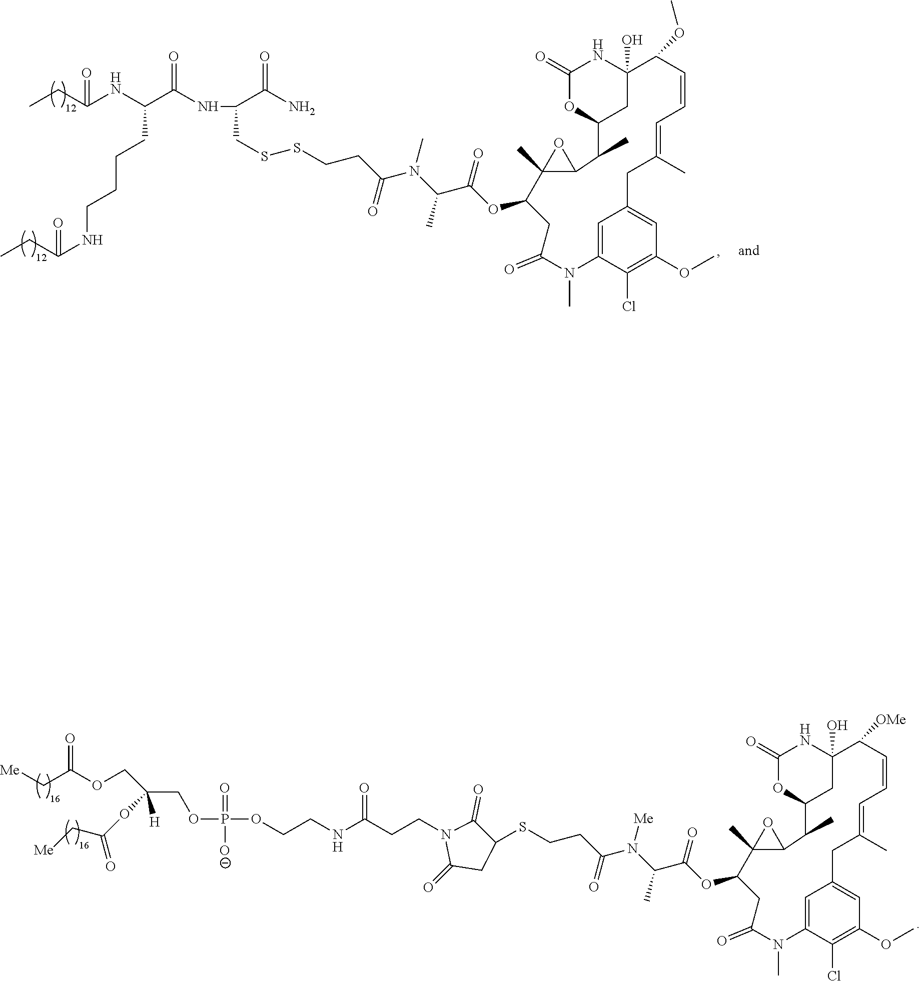

In some embodiments, the targeting moiety-lipid conjugate and a lipid of the prodrug-lipid conjugate comprise a C₁₄-C₁₈ fatty acid. In various embodiments, the peptide comprises an amino acid sequence of RKRLQVQLSIRT (SEQ ID NO: 1).

In some embodiments, the lipid of the targeting moiety-lipid conjugate comprises a C₁₆ fatty acid. In some embodiments, the prodrug comprises a C₁₄, a C₁₆, or a C₁₈ fatty acid. In some embodiments, the C₁₄ fatty acid is myristic acid, the C₁₆ fatty acid is palmitic acid, and the Cis fatty acid is stearic acid.

In some embodiments, the polyethylene glycol-lipid conjugate is 1,2-distearoyl-sn-glycero-3-phosphoethanolamine-N-[methoxy(polyethylene glycol)-2000](ammonium salt) (mPEG2000-DSPE). In various embodiments, the bulk lipid comprises 1,2-distearoyl-sn-glycero-3-phosphocholine (DSPC).

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following drawings form part of the specification and are included to further demonstrate embodiments or various aspects of the present disclosure. In some instances, embodiments of the disclosure can be best understood by referring to the accompanying drawings in combination with the detailed description presented herein. The description and accompanying drawings may highlight a certain specific example, or a certain aspect of the disclosure. However, one skilled in the art will understand that portions of the example or aspect may be used in combination with other examples or aspects of the invention.

FIG. 1. Selective DM1-Prodrug release mechanism from targeted liposomal nanoparticles into the cellular environment. Illustration of targeted liposomal nanoparticle containing prodrug (TNP[Prodrug]) selectively targeting highly expressed receptors on target cell. TNP[Prodrug] gets internalized by receptor mediated endocytosis and thus delivers drug selectively into cytosolic compartment of target cell.

FIG. 2A-B. Linkage moieties designed and synthesized for the preparation of DM1-Prodrug molecules. a) Schematic of DM1-Prodrug synthesis. b) Structure of synthesized linkage moieties for DM1-Prodrug formulation. Linkage moiety # for DM1-Prodrugs: (1) R=DM1-Amide-C14; (2) R=DM1-Ester-C14; (3) R=DM1-SS-C14; (4) R=DM1-DSPE.

FIG. 3A-C. Preparation and characterization of liposomal nanoparticles. a) Synthesis of different DM1-Prodrug molecules using lipid-conjugate versions with linkage moieties of different chemistry. b) HPLC chromatogram of a pure Prodrug-4 vs. Free DM1 using C₃ column. c) Schematic of preparing targeted liposomal DM1-Prodrug (TNP[Prodrug]) using bulk lipid, PEG-lipid conjugate, sterol (e.g., cholesterol), CD138pep-lipid, and DM1-Prodrug at specific stoichiometry via hydration and extrusion.

FIG. 4A-F. In vitro cytotoxicity evaluation of liposomal prodrug formulations. a)-e) NCI—H929 MM Cells were cultured in the presence of equivalent DM1 concentrations of NP[Prodrug], TNP[Prodrug], free DM1-Prodrug, or free DM1 for 24 h, 48 h and 72 h. Cell viability was assessed by CCK-8. Each data represents means of triplicate cultures (±s.d.). f) Summary of average IC₅₀ for different DM1-Prodrug formulations and fold enhancements of TNP formulations over NP formulations.

FIG. 5A-B. Maximum tolerated dose (MTD) study with NP[Prodrug-4] formulation for different dose of equivalent free drug. The maximum tolerated dose for both free DM1 and liposomal Prodrug-4 formulations were determined by evaluating them on healthy NOD-SCID mice. a) Mice lost over 15% of their body weight and were all morbid before the end of the week, when they were injected with free DM1 at a dose over 0.5 mg/kg twice on days 0 and 3. b) Negligible toxicity was observed when mice were injected using NP[Prodrug-4] formulations at various equivalent DM1 concentrations on days 0 and 3. n=4-5 for all groups and data represents means (±s.d.).

FIG. 6A-G. In vivo efficacy evaluation of liposomal Prodrug-4 formulation. Subcutaneous xenograft NOD-SCID mouse model was used to test NP[Prodrug-4] and TNP[Prodrug-4] treatment. Mice were injected with H929 cells and tumors were allowed to grow to a predetermined size prior to treatments. Mice were injected with PBS (control), NP[Prodrug-4] or TNP[Prodrug-4] on indicated days (a). Tumor volume (b, c), systemic toxicity (d, e), and survival (f) were tracked during 18 days of the study period daily for treatments. At the end of the study, organs were dissected and weighed (g). n=5-6 for all groups and data represents means (±s.e.). Unpaired t-test was used for determining P-value (**, p<0.0001; *, p<0.05).

FIG. 7. Targeting CD138pep-lipid construct to make liposomal prodrug. Design of CD138 targeting peptide-lipid conjugate contains ethylene glycol (EG) spacer, oligolysine (K₃) content, EG₆ peptide-linker and palmitic acid lipid tails.

FIG. 8A-D. a) Synthesis Schematic of DM1-Amide-C14 (Prodrug-1). DM1 and MPS—COOH were reacted at 1:1.2 mol ratio using DMF at room temperature overnight, followed by purification using C3 semiprep column via RP-HPLC. The C14-R—NH₂ was synthesized on solid support using Fmoc chemistry and purified by normal phase micropipette column. Both pure conjugates were reacted together in DMF, along with the addition of HBTU and DIEA for the synthesis of final product DM1-Amide-C14. b) Synthesis Schematic of DM1-Ester-C14 (Prodrug-2). DM1 and MPS—COOH were reacted at 1:1.2 mol ratio using DMF at room temperature overnight, followed by purification using C3 semiprep column via RP-HPLC. The C14-R—OH was synthesized on solid support using Fmoc chemistry and purified by normal phase micropipette column. Both pure conjugates were reacted together in anhydrous DCM, along with the addition of 5% DMAP and EDC for the synthesis of final product DM1-Ester-C14. c) Synthesis Schematic of DM1-SS-C14 (Prodrug-3). The C14-R-SH was synthesized on solid support using Fmoc chemistry and purified by normal phase micropipette column. Then the pure conjugate and DM1 were reacted together in DMF at room temperature overnight for the synthesis of the final product DM1-SS-C14, followed by purification using C3 semiprep column via RP-HPLC. d) Synthesis Schematic of DM1-DSPE (Prodrug-4). DM1 and DSPE-maleimide were reacted at 1:1.2 mol ratio using DMF at room temperature overnight, followed by purification using C3 semiprep column via RP-HPLC.

FIG. 9. Zeta potential of nanoparticles. Analysis of nanoparticle formulations was performed at pH 7.4 as described in the methods section.

FIG. 10. DM1-Prodrug loading efficiency in liposomal nanoparticles. The loading efficiency of DM1-Prodrug is critical for maintaining the precise molar prodrug ratio and for minimizing variability during nanoparticle formation. To maintain high loading efficiencies, 5 mol % drug loading was selected to be incorporated with nanoparticles, which yielded loading efficiencies of >91% at 37° C. in PBS. Briefly, nanoparticles were prepared using 5 mol % of prodrug along with other liposomal components. Then, nanoparticles were further purified using LEP and analyzed for their ability to retain prodrug using a Zorbax C3 semiprep column and a 2-propanol/acetonitrile/water gradient. Prodrug retention=Post-LEP AUC of Prodrug Peak/Pre-LEP AUC of Prodrug Peak.

FIG. 11. NP[Prodrug] stability in phosphate buffer (PB), acetate buffer (AB) and PB containing enzymes (Enzyme cocktail) at 37° C. Briefly, nanoparticles were prepared and incubated in buffers for 0, 24, and 48 h at 37° C., while the samples were constantly shaking. Nanoparticles were analyzed after incubation period to determine whether prodrug molecules were stable under different pH and reaction condition, using a Zorbax C3 semiprep column and a 2-propanol/acetonitrile/water gradient. Normalized AUC=(AUC of Prodrug Peak at a given hour/AUC of Prodrug Peak at 0 h). PB and AB had pH values of 7.4 and pH 4.8 respectively; while enzyme cocktail had a pH of 7.4 to facilitate enzymatic reaction.

FIG. 12A-F. In vitro cytotoxicity evaluation of free drug and free DM1-Prodrug. a)-e) NCI-H929 MM Cells were cultured in the presence of equivalent DM1 concentrations of free DM1-Prodrug, or free DM1 for 24 h, 48 h and 72 h. Cell viability was assessed by CCK-8. Each data represents means of triplicate cultures (±s.d.). f) Summary of average IC₅₀ for free DM1-Prodrug and free DM1.

FIG. 13. Enhanced cellular binding & uptake of CD138 targeted nanoparticles (TNP) over non-targeted nanoparticles (NP). Flow cytometry analysis of cellular binding and uptake of CD138pep TNP on human multiple myeloma NCI-H929 cell line. CD138-TNP exhibited ~491-fold and ~107-fold enhanced binding and uptake respectively compared to NPs. Cells were incubated with DiO labeled nanoparticles. Furthermore, nanoparticles were incorporated with or without Prodrug-4 and then binding and uptake were compared. In this case, Prodrug-4 was chosen due to its higher in vitro potency. As expected, prodrug-loaded nanoparticles had no effect on the binding and uptake of cells compared to the empty CD138-TNPs. All data represents means (±SD) of triplicate cultures. RFU, relative fluorescence unit; N.S., non-significant.

FIG. 14A-B. In vivo efficacy evaluation of liposomal Prodrug-4 formulation by delivering 1 mg/kg equivalent of DM1. Subcutaneous xenograft NOD-SCID mouse model was used to test NP[Prodrug-4] and TNP[Prodrug-4] treatment. Mice were injected with H929 cells and tumors allowed to grow to a predetermined size prior to treatments. Mice were injected with PBS (control), NP[Prodrug-4] or TNP[Prodrug-4] on indicated days. At the end of the study, organs were dissected and weighed (a). Survival (b) was tracked during 18 days of study period daily. n=5-6 for all groups and data represents means (±s.e.). Unpaired t-test was used for P value (*, p<0.05).

FIG. 15. In vitro cytotoxicity assessment of intermediate compound DM1-COOH at 48 h using CCK-8. The potency of Prodrug-2 might have shifted compared to the other versions. The exact mechanism is very hard to determine, however, our hypothesis is the resulting negative charge on the “active” drug molecule, and the position of this charge, subsequent to linker hydrolysis is affecting the molecule's activity in a strongly negative manner. Upon hydrolysis of Prodrug-2, the resulting structure of the “active drug” would be identical to the structure of DM1-COOH (FIG. 8b) and is responsible for the shift in IC₅₀. To confirm our prediction, we performed a cytotoxicity assay with the intermediate compound DM1-COOH and observed a barely noticeable reduction in cell viability. This observation demonstrates the detrimental effects of a negative charge on the mechanistic activity of the DM1 drug molecule.

### DETAILED DESCRIPTION

### Definitions

In the following detailed description, reference is made to the accompanying drawings that form a part hereof and that are shown by way of illustration embodiments that may be practiced. It is to be understood that other embodiments may be utilized and structural or logical changes may be made without departing from the scope. Thus, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use the terms “embodiment” or “embodiments,” which may each refer to one or more of the same or different embodiments. Furthermore, the terms “comprising,” “including,” “having,” and the like, as used with respect to embodiments, are synonymous. The use of the word “a” or “an” when used in conjunction with the term “comprising” in the claims and/or the specification may mean “one,” but it is also consistent with the meaning of “one or more,” “at least one,” and “one or more than one.” Some embodiments of the present disclosure may consist of or consist essentially of one or more elements, method steps, and/or methods of the invention. It is contemplated that any method or composition described herein can be implemented with respect to any other method or composition described herein.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The term “contact”, as used herein, refers to an addition to or an interaction between, at least, two molecules, that causes an increase or decrease in the magnitude of a certain activity or function of the molecules compared to the magnitude of the activity or function observed in the absence of, at least, one of the molecules. Example includes, but is not limited to, contact of sera to cells in culture.

As used herein, “subject” refers to a person, an individual, or animal that is the object of medical or scientific study or a patient. In another aspect, the present disclosure provides a composition of matter and method of administrating said composition of matter to a subject, preferably a human, or in a format that can be diluted or reconstituted for administration to the subject.

As used herein, a “bulk lipid” is any compatible lipid that has a hydrophilic region and a hydrocarbon tail that can facilitate the incorporation of a drug-lipid conjugate into a lipid membrane. Examples include, but are not limited to, phospholipids, such as 1,2-distearoyl-sn-glycero-3-phosphocholine (DSPC), and fatty acids, such as, myristic acid.

As used herein, “nanoparticle” refers to any partially or wholly lipid-coated nanostructure having a cross-section length (“diameter”) in the range of 1 to 300 nanometers (nm). As used herein, cross-section length refers to the measurement of the longest cross-section length of the nanoparticle (e.g., the longest distance that can be measured between two points of a cross-section of the nanoparticle). In some instances, such particles will have a cross-section length in the range of about 10 nm to about 300 nm, about 10 to about 250 nm, about 10 to about 200 nm, about 10 to about 150 nm, about 50 to 125 nm, about 10 to about 120 nm, about 10 to 115 nm, about 10 to 110 nm, about 10 to 105 nm, about 10 to 100 nanometers, and/or 50 to 110 nm.

The terms “conjugate” and “conjugated” as used herein can refer to the attachment (e.g., the covalent attachment) of two or more components (e.g., chemical compounds, polymers, biomolecule, particles, etc.) to one another. In some embodiments, a conjugate can comprise monovalent moieties derived from two different chemical compounds covalently linked via a bivalent linker moiety (e.g., an optionally substituted alkylene or arylene). In some embodiments, the linker can contain one or more biodegradable bond, such that one or more bonds in the linker can be broken when the prodrug is exposed to a particular physiological environment or enzyme.

The term “prodrug” as used herein, can refer to a compound that, upon administration to a subject or sample, is capable of providing (directly or indirectly) another compound (i.e., a “parent compound”) having a desired biological activity (e.g., anticancer activity). In some, but not all, embodiments, the prodrug compound has less of the desired biological activity than the parent compound. In some embodiments, the prodrug compound has no measurable biological activity prior to transformation to the parent compound. In some embodiments, the prodrug itself has the desired activity.

Transformation of the prodrug to the parent compound can take place in the presence of particular enzymes (e.g., esterases) or under certain biological conditions (e.g., at a physiologically relevant pH or in the presence of reducing agents present in a physiological environment). In some embodiments, the prodrug is initially transformed into another prodrug, which is then transformed (sometimes much more slowly) into the parent compound. Prodrugs can provide increased bioavailability and/or enhanced delivery to a biological compartment (e.g., a lysosome, the brain or lymphatic system, etc.) relative to a parent compound. In some embodiments, the prodrug can be more compatible with a particular delivery platform or formulation than the parent compound.

### Embodiments of the Invention

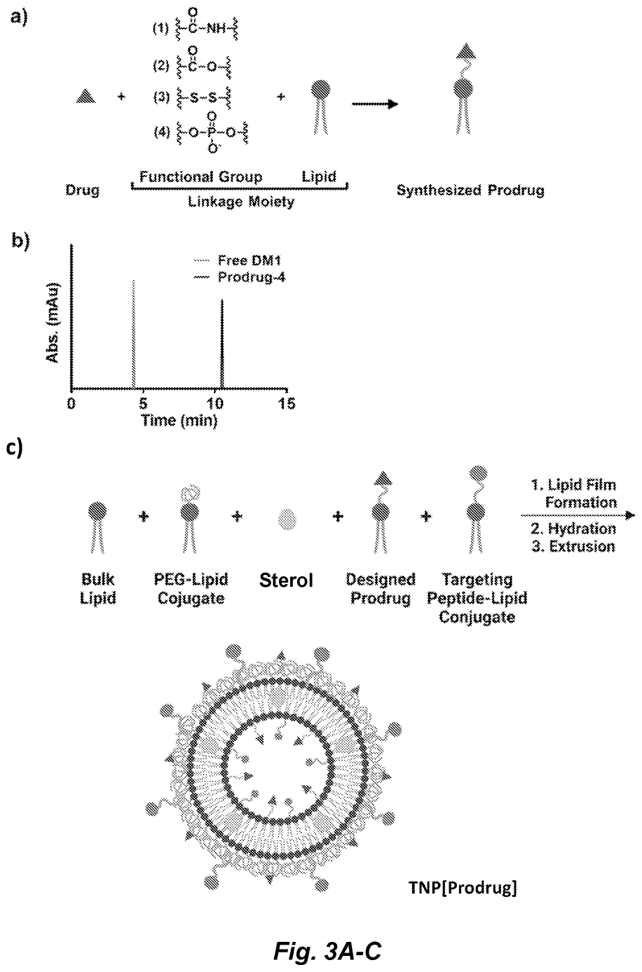

In some embodiments, a nanoparticle generally comprises a prodrug comprising a drug-lipid conjugate wherein a chemical linker group is positioned between a drug and a lipid of the drug-lipid conjugate; a targeting moiety-lipid conjugate; a polyethylene glycol-lipid conjugate; a bulk lipid; and optionally, cholesterol (see, for example, FIG. 3C).

A nanoparticle can be designed for use in in vitro and in vivo applications. Generally, a nanoparticle can have a hydrocarbon interior portion surrounded by an outer portion that includes a hydrophilic region. The hydrophobic core may be formed by hydrocarbon tails of the bulk lipids, PEG-lipid conjugates, the prodrug, and the targeting moiety-lipid conjugate. Typically, a portion of the prodrug and the targeting peptide may be oriented such that some of the targeting peptides and the drug portion of the drug-lipid conjugate are entrapped in the interior portion of the nanoparticle. In some embodiments, the hydrophilic region may be formed by water-soluble polymers (e.g., PEG). Preferably, the hydrophilic region is formed by a polyethylene glycol (PEG) region of a PEG-lipid conjugate.

In some embodiments, the prodrug comprises the formula A-B-C, where A is a drug moiety, B is a chemical linker group, and C is a lipid. In some embodiments, the lipid moiety of the prodrug comprises a C₁₄-C₁₈ fatty acid. In preferred embodiments, the C₁₄-C₁₈ fatty acid is a myristic acid moiety, a palmitic acid moiety, or a stearic acid moiety.

In some embodiments, the drug moiety of the drug-lipid conjugate comprises a chemotherapeutic agent covalently coupled to the lipid to form the prodrug. Exemplary chemotherapeutic agents include, for example, a platinum compound, paclitaxel; carboplatin; bortezomib; vorinostat; rituximab; temozolomide; rapamycin; an alkylating agent; cyclosphosphamide; an alkyl sulfonate; busulfan; improsulfan; piposulfan; an aziridine; an ethylenimine; a methylamelamine; an acetogenin; a camptothecin; a cryptophycin; a nitrogen mustard; a nitrosurea; an antibiotic; a enediyne antibiotic; a bisphosphonate; doxorubicin; a mitomycin; an anti-metabolite; a folic acid analogue; a purine analog; a pyrimidine analog; an androgen; an anti-adrenal; an epothilone; a trichothecene; gemcitabine; 6-thioguanine; mercaptopurine; methotrexate; vinblastine; etoposide; ifosfamide; mitoxantrone; vincristine; vinorelbine; novantrone; teniposide; edatrexate; daunomycin; aminopterin; xeloda; ibandronate; irinotecan; a topoisomerase inhibitor; a retinoid; capecitabine; combretastatin; leucovorin; lapatinib; erlotinib; and a maytansinoids.

The drug moiety can comprise a maytansinoid or other microtubule inhibitor. In some embodiments, the drug moiety comprises mertansine (DM1):

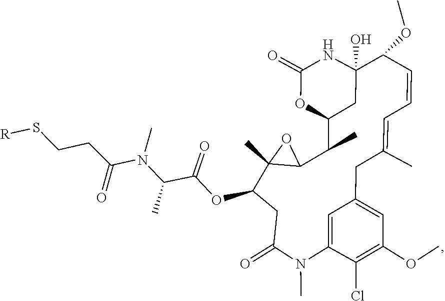

wherein R is the chemical linker group that links the drug moiety to the lipid of the drug-lipid conjugate. Preferably, the chemical linker group comprises one of an amide linker, an ester linker, a disulfide linker, and a phosphodiester linker, for example, a linking group (chemical linker group) of one of the following moieties (1)-(4).

In some embodiments, a) the lipid of the drug-lipid conjugate and the amide linker comprise moiety (1):

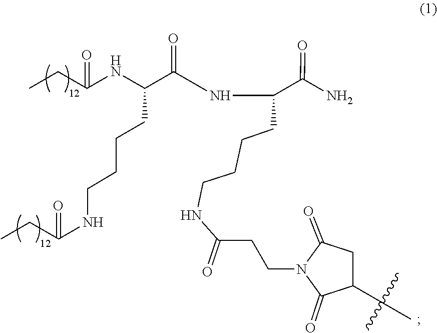

b) the lipid of the drug-lipid conjugate and the ester linker comprise moiety (2):

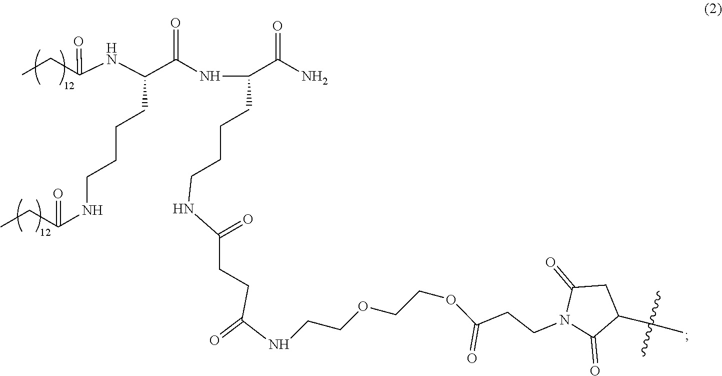

c) the lipid of the drug-lipid conjugate and the disulfide linker comprise moiety (3):

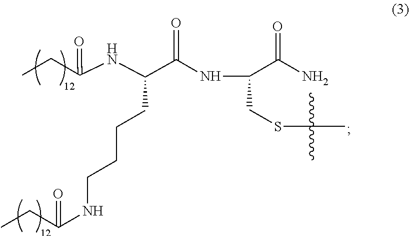

d) the lipid of the drug-lipid conjugate and the phosphodiester linker comprise moiety (4):

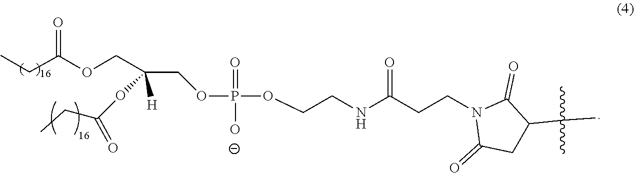

In various embodiments, the prodrug is one of the following:

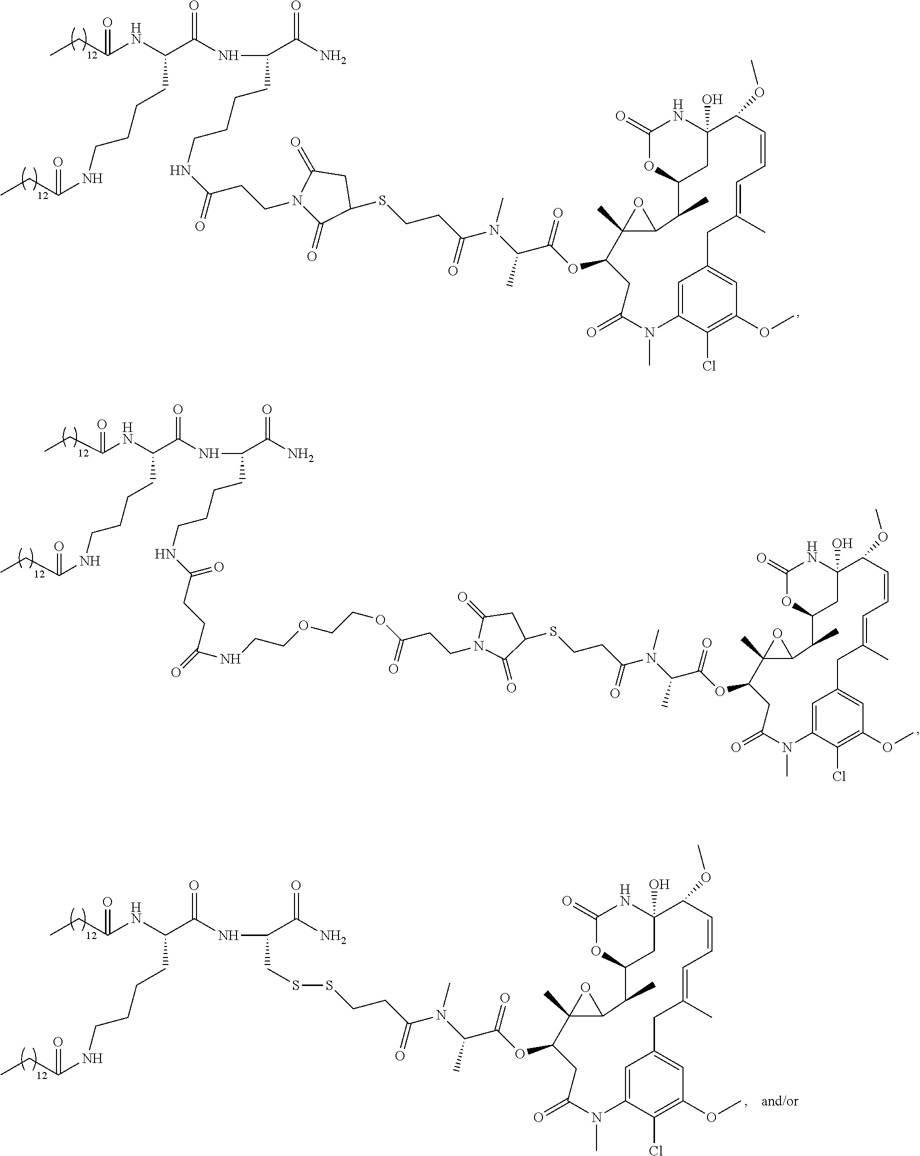

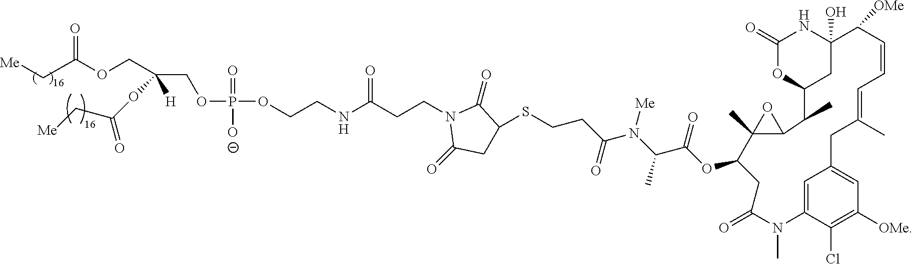

In some embodiments, a nanoparticle comprises only a single species of prodrug.

In some embodiments, the prodrug can comprise about 1 mol %, about 2 mol %, about 3 mol %, about 4 mol %, about 5 mol %, about 6 mol %, about 7 mol %, about 8 mol %, about 9 mol %, about 10 mol %, about 11 mol %, about 12 mol %, about 13 mol %, about 14 mol %, about 15 mol %, about 16 mol %, about 17 mol %, about 18 mol %, about 19 mol %, or about 20 mol % of the nanoparticle. In some embodiments, the prodrug can comprise about 1 mol % to about 10 mol % or about 1 mol % to about 5 mol % of a nanoparticle. In other embodiments, the prodrug can comprise about 0.1 mol % to about 1 mol % of a nanoparticle.

In some embodiments, the targeting moiety of the targeting moiety-lipid conjugate comprises an antibody, an antibody fragment, a peptide, a protein, or a ligand. Preferably, the targeting moiety specifically binds to a CD138 receptor displayed on the surface of a cell, and in particular, a cancerous cell. In some embodiments, the targeting moiety is a peptide that specifically binds to a CD138 receptor. In one embodiment, the targeting peptide comprises the amino acid sequence RKRLQVQLSIRT (SEQ ID NO: 1). In another embodiment, the targeting peptide consists of the amino acid sequence RKRLQVQLSIRT (SEQ ID NO: 1).

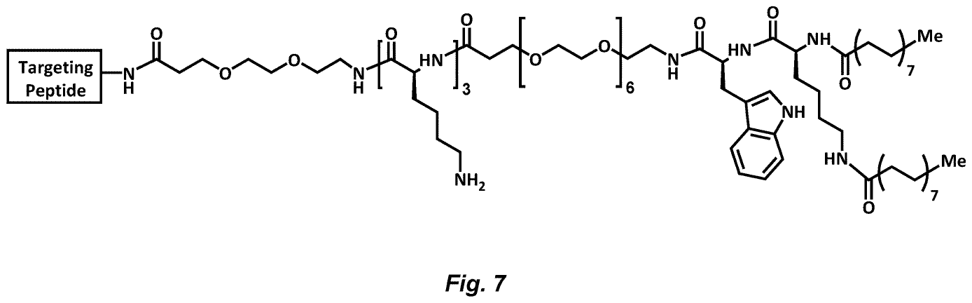

In some embodiments, the targeting moiety-lipid conjugate can comprise one or more linkers disposed between the targeting moiety and the lipid (see FIG. 7). As used herein, a “linker” can be a sugar, an oligosaccharide, an amino acid, peptides, a polymer, or other molecules that can provide favorable results in targeting peptide display and binding. Examples of a linker include, but are not limited to, ethylene glycol molecules (e.g., polyethylene glycol). In some embodiments, a linker comprises polyethylene glycol polymers. For example, the PEG linker may comprise about 1 to about 20 ethylene glycol residues, about 1 to about 15 ethylene glycol residues, or about 1 to about 10 ethylene glycol residues. In one embodiment, the PEG linker comprises about 2 to 6 ethylene glycol residues.

In other embodiments, the linker can be any moiety that will improve targeting peptide-lipid water solubility profile. The linker increases hydrophilicity and improves targeting peptide display on the nanoparticle surface. Examples include, but are not limited to, charged amino acids such as aspartic acid (D), glutamic acid (E), lysine (K) and arginine (R) or polar amino acids, such as, glutamine (Q), asparagine (N), histidine (H), serine (S), threonine (T), and methionine (M). In some embodiments, an amino acid may comprise one or more amino acids, and in particular, one or more charged amino acids such as poly-lysine (e.g., a monomer, dimer or trimer).

In some embodiments, the linker may comprise a polymer of ethylene glycol attached to a peptide. For example, the ethylene glycol polymer may comprise about 1 to about 20 ethylene glycol residues, or about 6 to about 18 ethylene glycol residues, and the peptide may comprise an amino acid such as a tryptophane residue or a monomer, dimer, or trimer of tryptophan or lysine.

In some embodiments, the linker comprises the formula D-E-F, wherein D is a polymer of ethylene glycol residues, E is one or more lysine residues, and F is a peptide-ethylene glycol polymer comprising a tryptophan residue and a plurality of ethylene glycol residues, wherein D is conjugated to the targeting moiety and F is conjugated to the lipid.

In one certain embodiment, the linker comprises the formula D-E-F, wherein D is about 2 ethylene glycol residues, E is about 3 lysine residues, and F is a peptide-ethylene glycol comprising a tryptophan residue and about 6 ethylene glycol residues, wherein D is further conjugated to the targeting moiety and F is conjugated to the lipid.

In some embodiments, the targeting moiety-lipid conjugate can comprise about 1 mol %, about 2 mol %, about 3 mol %, about 4 mol %, about 5 mol %, about 6 mol %, about 7 mol %, about 8 mol %, about 9 mol %, about 10 mol %, about 11 mol %, about 12 mol %, about 13 mol %, about 14 mol %, about 15 mol %, about 16 mol %, about 17 mol %, about 18 mol %, about 19 mol %, or about 20 mol % of the nanoparticle. In some embodiments, the targeting moiety-lipid conjugate can comprise about 1 mol % to about 10 mol % or about 1 mol % to about 5 mol % of a nanoparticle.

Bulk lipids can include a lipid molecule coupled directly or indirectly to one or more additional molecules, or just lipid molecules. Typically, lipid molecules are amphipathic lipid molecules, each with a polar/hydrophilic region and a non-polar/hydrophobic/hydrocarbon tail. Optionally, some or all of the lipid molecules can be phospholipids or fatty acids or compatible lipids that can facilitate the components incorporation into a lipid membrane.

Exemplary phospholipids, include, but are not limited to, phosphatidyl cholines; phosphatidyl cholines with acyl groups having 6 to 22 carbon atoms; phosphatidyl ethanolamines; phosphatidyl inositols; phosphatidic acids; phosphatidyl serines; sphingomyelin; phosphatidyl glycerols; phosphatidylcholine; phosphatidylglycerol; lecithin; β,γ-dipalmitoyl-α-lecithin; sphingomyelin; phosphatidylserine; phosphatidic acid; N-(2,3-di(9-(Z)-octadecenyloxy))-prop-1-yl-N,N,N-trimethylammonium chloride; phosphatidylethanolamine; lysolecithin; lysophosphatidylethanolamine; phosphatidylinositol; cephalin; cardiolipin; cerebrosides; dicetylphosphate; dioleoylphosphatidylcholine; dipalmitoylphosphatidylcholine; dipalmitoylphosphatidylglycerol; dioleoylphosphatidylglycerol; palmitoyl-oleoyl-phosphatidylcholine; di-stearoyl-phosphatidylcholine; stearoyl-palmitoyl-phosphatidylcholine; di-palmitoyl-phosphatidylethanolamine; di-stearoyl-phosphatidylethanolamine; di-myrstoyl-phosphatidylserine; di-oleyl-phosphatidylcholine; dimyristoyl phosphatidyl choline (DMPC); dioleoylphosphatidylethanolamine (DOPE); palmitoyloleoylphosphatidylcholine (POPC); egg phosphatidylcholine (EPC); distearoylphosphatidylcholine (DSPC); dioleoylphosphatidylcholine (DOPC); dipalmitoylphosphatidylcholine (DPPC); dioleoylphosphatidylglycerol (DOPG); dipalmitoylphosphatidylglycerol (DPPG); -phosphatidylethanolamine (POPE); dioleoyl-phosphatidylethanolamine 4-(N-maleimidomethyl)-cyclohexane-1-carboxylate (DOPE-mal); L-a-phosphatidylcholine; and any combinations thereof. Exemplary fatty acids include palmitic acid, myristic acid, palmitic acid, and stearic acid. In some embodiments, the bulk lipid comprises 1,2-distearoyl-sn-glycero-3-phosphocholine (DSPC).

In some embodiments, the bulk lipid may include about 60 mol %, 61 mol %, about 62 mol %, about 63 mol %, 64 mol %, 65 mol %, about 66 mol %, about 67 mol %, 68 mol %, 69 mol %, about 70 mol %, about 71 mol %, about 72 mol %, about 73 mol %, about 74 mol %, about 75 mol %, about 76 mol %, about 77 mol %, about 78 mol %, about 79 mol %, about 80 mol %, about 81 mol %, about 82 mol %, about 83 mol %, 84 mol %, about 85 mol %, about 86 mol %, about 87 mol %, 88 mol %, about 89 mol %, about 90 mol %, about 91 mol %, about 92 mol %, about 93 mol %, about 94 mol %, about 95 mol %, about 96 mol %, about 97 mol %, or greater than 97 mol % of the nanoparticle.

In some embodiments, nanoparticles may include a hydrophilic polymer conjugated to a hydrophobic region of lipid molecule. The polymer can be water-soluble polymer, such as polyethylene glycol (PEG), forming a PEG-lipid conjugate. In some embodiments, the PEG-lipid conjugates, comprises, for example, PEG conjugated diacylglycerols and dialkylglycerols; PEG-conjugated phosphatidylethanolamine and phosphatidic acid; PEG conjugated ceramides; PEG conjugated dialkylamines; PEG conjugated 1,2-diacyloxypropan-3-amines; 1,2-distearoyl-sn-glycem-3-phosphoethanolamine-N-[amino(polyethylene glycol)-2000](DSPE-PEG2000); and any combinations thereof. In some embodiments, the PEG-lipid conjugate comprises DSPE-PEG2000.

In some embodiments, the PEG-lipid conjugate can comprise about 1 mol %, about 2 mol %, about 3 mol %, about 4 mol %, about 5 mol %, about 6 mol %, about 7 mol %, about 8 mol %, about 9 mol %, about 10 mol %, about 11 mol %, about 12 mol %, about 13 mol %, about 14 mol %, about 15 mol %, about 16 mol %, about 17 mol %, about 18 mol %, about 19 mol %, or about 20 mol % of the nanoparticle. In some embodiments, the PEG-lipid conjugate can comprise about 1 mol % to about 10 mol % or about 1 mol % to about 5 mol % of a nanoparticle.

In some embodiments, a nanoparticle can include a molecule that can improve stability of the nanoparticle, such as, but is not limited to a sterol such as cholesterol, cholesterol-sulfate, a sterol-ester such as an ester linked fatty acids (C₁₆:0, C₁₈:1, and C₁₈:2) (e.g., cholesterol-palmitate), beta-sitosterol, stigmasterol, campesterol, lanosterol, brassicasterol, fucosterol, lathosterol, spinasterol, desmosterol, and dehydocholesterol, (e.g., 7-dehydrocholesterol). In some embodiments, the amount of a sterol (e.g., cholesterol) in the nanoparticle in an amount of about 0.1 mol % to about 10 mol %. In some embodiments, the amount of sterol (e.g., cholesterol) in the nanoparticle is about 1 mol % to about 5 mol %, or the amount of sterol (e.g., cholesterol) in the nanoparticle is about 5 mol %. In other embodiments, the nanoparticle does not include a sterol (e.g., cholesterol).

In some specific embodiments, the nanoparticle has a diameter of about 10 nm to about 300 nm. In other specific embodiments, the nanoparticle can have a diameter of about 80 nm to about 220 nm, about 100 nm to about 160 nm, or about 100 nm.

In some embodiments, a “molecular ratio” may be provided to indicate the number of molecules of two or more components in a nanoparticle. The number of molecules of a component in a nanoparticle may also be described in terms of a “mole percentage,” which is calculated by dividing the number of molecules of that component by the number of molecules in the nanoparticle. For example, in a nanoparticle with 100 component molecules, 69 of which are bulk lipid, 15 of which are prodrug, 10 of which are PEG-lipid conjugate, 5 of which is the targeting moiety-lipid conjugate, and 1 of which is cholesterol, then the “molecular ratio” of the components is 69:15:10:5:1, and the mole percentages of the three components are 69%, 15%, 10%, 5% and 1%, respectively. If not specifically identified, percentages referenced herein are molar percentages (mol %), unless the context specifically indicates otherwise.

Accordingly, in some embodiments, an amount of components of an exemplary a nanoparticle includes about 65 mol % to about 97 mol % bulk lipid, about 1 mol % to about 10 mol % PEG-lipid conjugate, about 1 mol % to about 10 mol % cholesterol, about 1 mol % to about 10 mol % prodrug, and about 0.01% to about 5 mol % targeting moiety-lipid conjugate.

In some embodiments, an amount of components of an exemplary a nanoparticle includes about 84 mol % to about 97 mol % bulk lipid, about 1 mol % to about 5 mol % PEG-lipid conjugate, about 1 mol % to about 5 mol % cholesterol, about 1 mol % to about 5 mol % prodrug, and about 0.01% to about 1 mol % targeting moiety-lipid conjugate.

In one certain embodiment, an amount of components of an exemplary a nanoparticle includes about 83 mol % to about 85 mol % bulk lipid, about 5 mol % PEG-lipid conjugate, about 5 mol % cholesterol, about 5 mol % prodrug, and about 0.01% to about 1 mol % targeting moiety-lipid conjugate, where the bulk lipid is DSPC, the PEG-lipid conjugate is PEG-DSPE, cholesterol, the prodrug is DM1-lipid, and the targeting moiety-lipid conjugate is CD138 peptide-lipid.

The disclosure also provides for composition comprising a nanoparticle as described herein and a pharmaceutically acceptable carrier, excipient, or diluent.

The disclosure also provides for methods of treating a cancer comprising administering to a subject having said cancer and effective amount of a nanoparticle, wherein the nanoparticle treats the cancer, wherein the nanoparticle comprises a prodrug comprising a drug-lipid conjugate, wherein a chemical linker group is positioned between a drug and a lipid of the drug-lipid conjugate; a targeting moiety-lipid conjugate; a polyethylene glycol-lipid conjugate, cholesterol; and a bulk lipid.

In some embodiments of a method of treating a cancer, the administered nanoparticle comprises about 1 mol % to about 10 mol % of the prodrug, about 0.01 mol % to about 10 mol % of the targeting moiety-lipid conjugate, about 1 mol % to about 10 mol % of the polyethylene glycol-lipid conjugate, about 1 mol % to about 10 mol % cholesterol, and about 60 mol % to about 97 mol % bulk lipid.

In some embodiments, a nanoparticle that may be used to treat a cancer includes a prodrug comprising one or more of:

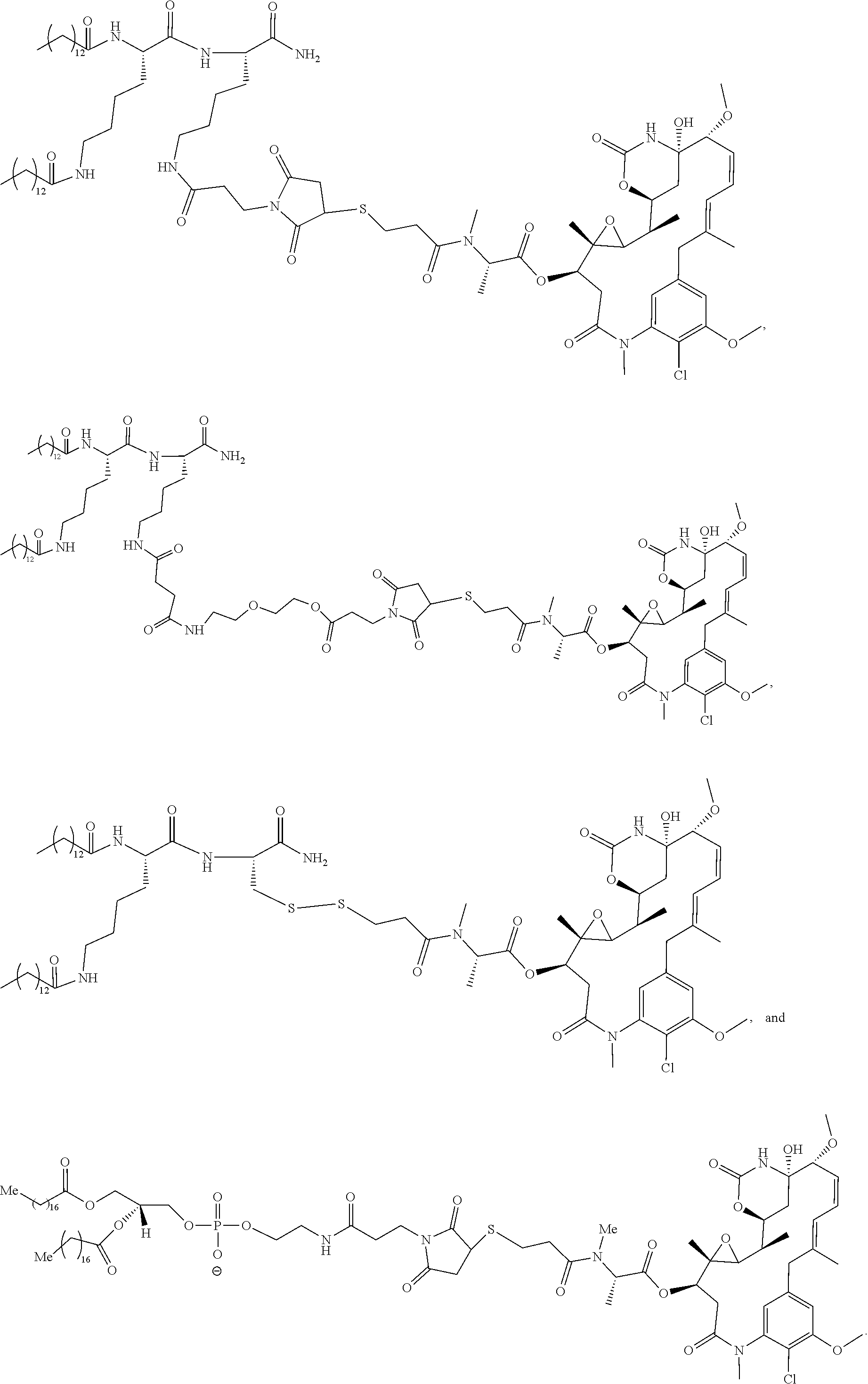

In some embodiments, the route of administration of the nanoparticle or pharmaceutical composition comprising the nanoparticle may include subcutaneous injection, intravenous injection or infusion, intramuscular injection, intraarterial administration, intrathecal administration, oral administration, sublingual administration, nasal administration, inhalation administration, rectal administration, or transdermal administration.

In some embodiments, an amount of about 0.1 mg/kg, about 0.2 mg/kg, about 0.3 mg/kg, about 0.4 mg/kg, about 0.5 mg·kg, about 0.6 mg/kg, about 0.7 mg/kg, about 0.8 mg/kg, about 0.9 mg/kg, about 1 mg/kg, about 1.25 mg/kg, about 1.5 mg/kg, about 1.75 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg. In some embodiments, the nanoparticles are administered over the course of a defined time period that may be consecutive or non-consecutive days. For example, doses may be administered on non-consecutive days over a time period of 10 days to about 25 days.

In some embodiments, the cancer comprises adipose cancer, anogenital cancer, breast cancer, bladder cancer, blood cancer, bone cancer, a brain tumor, central nervous system cancer, colon cancer, colorectal cancer, connective tissue cancer, a gynecological tumor, a head tumor, kidney cancer, lung cancer, lymphoid cancer, mesothelioma, multiple myeloma, a neck tumor, neuroblastoma, pancreatic cancer, prostate cancer, retinal cancer, skin cancer (e.g., melanoma), a soft tissue sarcoma, or stomach cancer. In certain specific embodiments, the cancer is multiple myeloma.

### Pharmaceutical Formulations.

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard- or soft-shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze-drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. No. 4,992,478 to Gerai et al., U.S. Pat. No. 4,820,508 to Wortzman et al., U.S. Pat. No. 4,608,392 to Jacquet et al., and U.S. Pat. No. 4,559,157 to Smith et al. Such dermatological compositions can be used in combinations with the compounds described herein where an ingredient of such compositions can optionally be replaced by a compound described herein, or a compound described herein can be added to the composition.

Useful dosages of the compositions described herein can be determined by comparing their in vitro activity, and in vivo activity in animal models. Methods for the extrapolation of effective dosages in mice, and other animals, to humans are known to the art; for example, see U.S. Pat. No. 4,938,949 to Borch et al. The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day.

The sub-dose itself may be further divided, e.g., into a number of discrete loosely spaced administrations, such as multiple inhalations from an insufflator or by application of a plurality of drops into the eye.

The compounds described herein can be effective anti-tumor agents and have higher potency and/or reduced toxicity as compared to known treatments for AML. Preferably, compounds of the invention are more potent and less toxic than known treatments, and/or avoid a potential site of catabolic metabolism encountered with known treatments, i.e., have a different metabolic profile than known treatments.

The invention provides therapeutic methods of treating cancer in a mammal, which involve administering to a mammal having cancer an effective amount of a compound or composition described herein. A mammal includes a primate, human, rodent, canine, feline, bovine, ovine, equine, swine, caprine, bovine and the like.

The ability of a compound of the invention to treat cancer may be determined by using assays well known to the art. For example, the design of treatment protocols, toxicity evaluation, data analysis, quantification of tumor cell-kill, and the biological significance of the use of transplantable tumor screens are known. In addition, ability of a compound to treat cancer may be determined using a tests as described herein or according to the test of a document cited herein.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Peptide-Targeted DM1 Loaded Liposomal Nanoparticles for Enhanced Efficacy in Treating Cancers

DM1, a derivative of naturally occurring maytansinoid toxin, induces cell cycle arrest by inhibiting the microtubule assembly. It has a narrow therapeutic window for oncology, nevertheless, its linkage to trastuzumab with a drug-antibody ratio [DAR] of 3.5, selectively targets malignant cells that overexpress the HER2, thereby widening its therapeutic window. DM1 contains thiol moiety playing an essential role in synthesizing antibody-drug conjugates, which we also utilized in our prodrug synthesis.

Some of the most common functional groups that are utilized in prodrug design include carboxylic, hydroxyl, amine, phosphate/phosphonate, and carbonyl groups. In most cases, prodrugs require only one or two chemical or enzymatic reaction steps to yield the active parent drug. For instance, in prodrug designs, amides are commonly used because of their relatively high enzymatic stability in vivo. Amides are generally hydrolyzed by ubiquitous amidases, peptidases, or proteases. Phosphate functional groups are utilized typically to improve the aqueous solubility of poorly water-soluble drugs in order to get favorable oral or parenteral administration. Typically, the presence of a dianionic phosphate moiety increases water solubility. Prodrugs containing phosphates demonstrate excellent stability and rapid bioconversion back to the parent drug by phosphatases. They are hydrolyzed by alkaline phosphatases at a similar rate by different species used in preclinical models. Esters are also another common functional group in prodrug design. Once in the systemic circulation, an ester bond is readily hydrolyzed by ubiquitous esterases found in the blood, liver, and other organs and tissues. Almost 50% of all marketed prodrugs are activated by enzymatic hydrolysis.

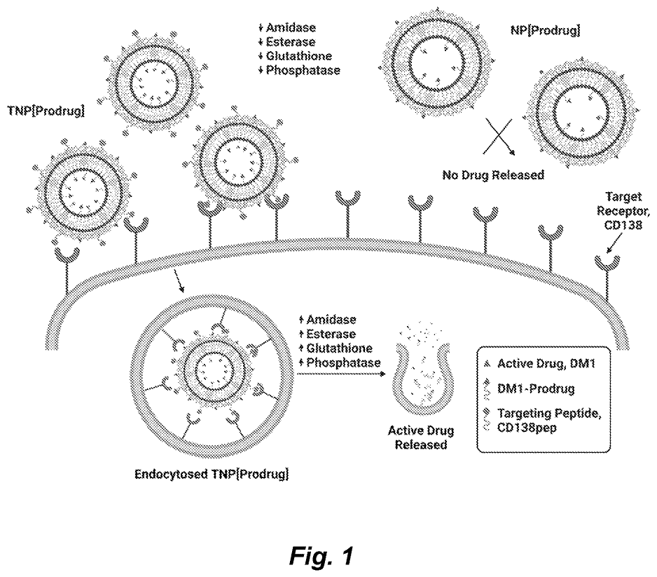

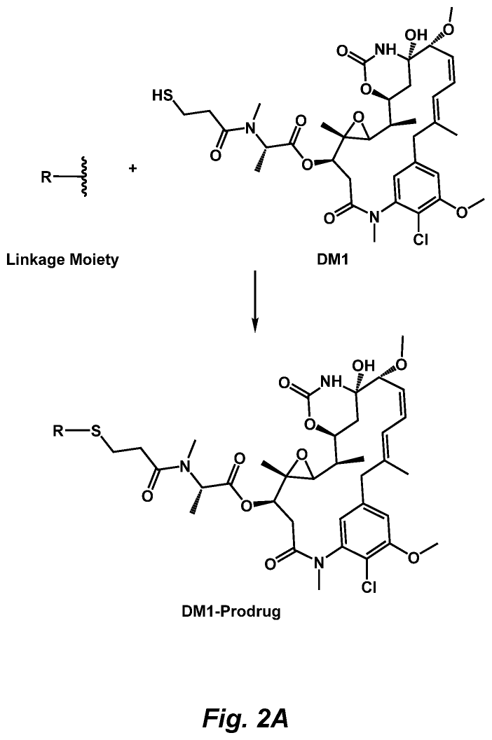

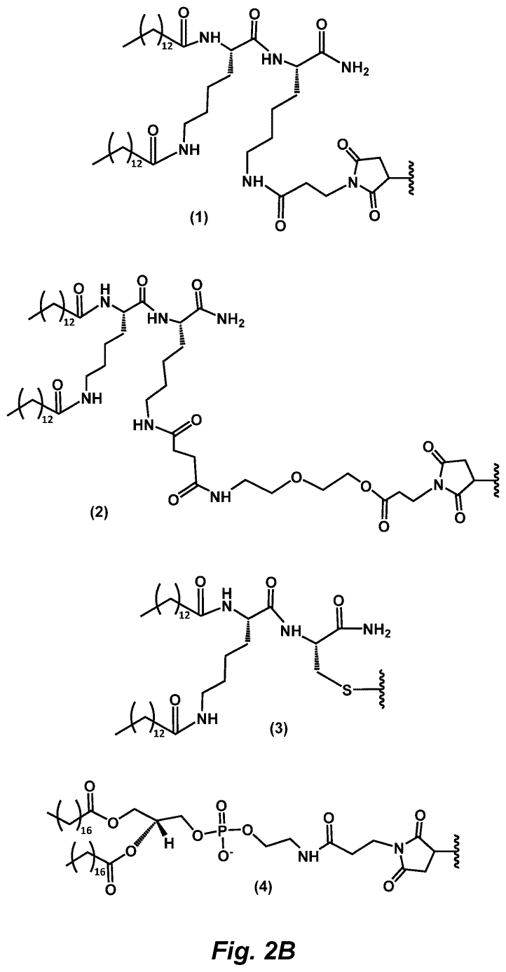

We designed several linkage moieties to make four different DM1-Prodrug molecules, containing either amide, ester, disulfide, or phosphodiester functional group, that undergoes enzymatic/chemical reaction in cellular environment and then release the active drug DM1 (FIG. 1). The linkage moieties are conjugated to DM1 via a thioether functional group (FIG. 2a). Pure liposomal DM1-Prodrug can be prepared by incorporating the DM1-Prodrug molecule along with other liposomal components to provide controlled drug release. To evaluate the significance and susceptibility of different functional groups between the active drug and its linkage moieties, we developed four DM1-Prodrug molecules as depicted in FIG. 2b.

It is broadly accepted that liposomal nanoparticles preferentially accumulate at the tumor site via enhanced permeability and retention (EPR), and active transport effect. To enhance the endocytosis of liposomes by the target cancer cells for effective cellular delivery, an additional design element is introduced into nanoparticles by the incorporation of surface functionalization with peptide ligands to achieve active targeting. Due to its significance in multiple myeloma (MM), we selected CD138 as a viable receptor to target as part of active delivery of this particular drug, DM1. CD138 is a surface antigen overexpressed in MM cells, and currently there is an anti-CD138 monoclonal antibody (Indatuximab Ravtansine) therapeutic available in Phase II clinical trial, emphasizing the importance of this receptor in MM treatment. Consequently, we developed TNP[Prodrug] by utilizing the previously reported peptide ligand CD138pep (RKRLQVQLSIRT) as the targeting element (FIG. 7). We settled on using 1% CD138pep loading in vitro to identify nanoparticle formulations of DM1, since maximum cellular uptake would help in narrowing down the most potent DM1-Prodrug. For in vivo evaluation, liposomes were loaded, with 0.1% CD138pep to achieve highest overall efficacy, since a study we reported earlier had identified that using a higher loading resulted in substantial reduction in tumor accumulation of the targeted particles.

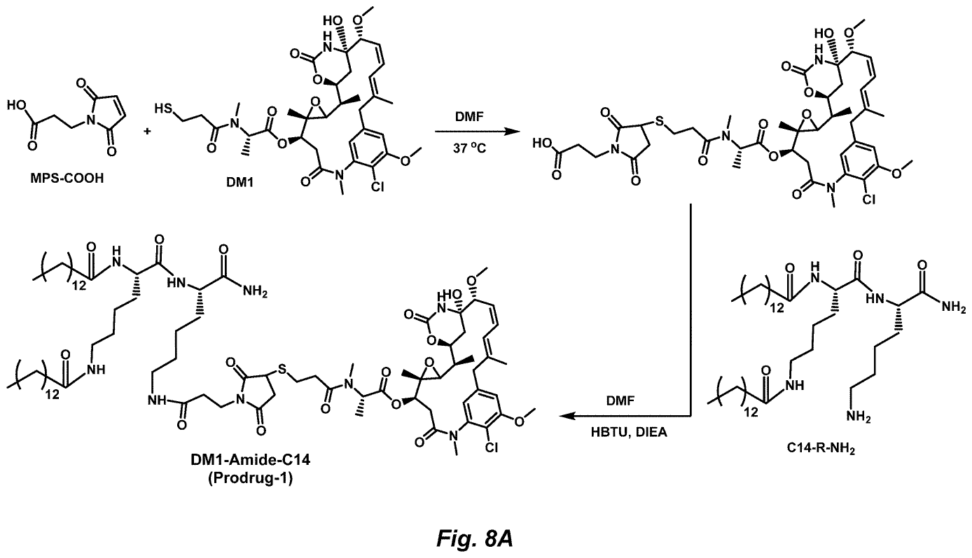

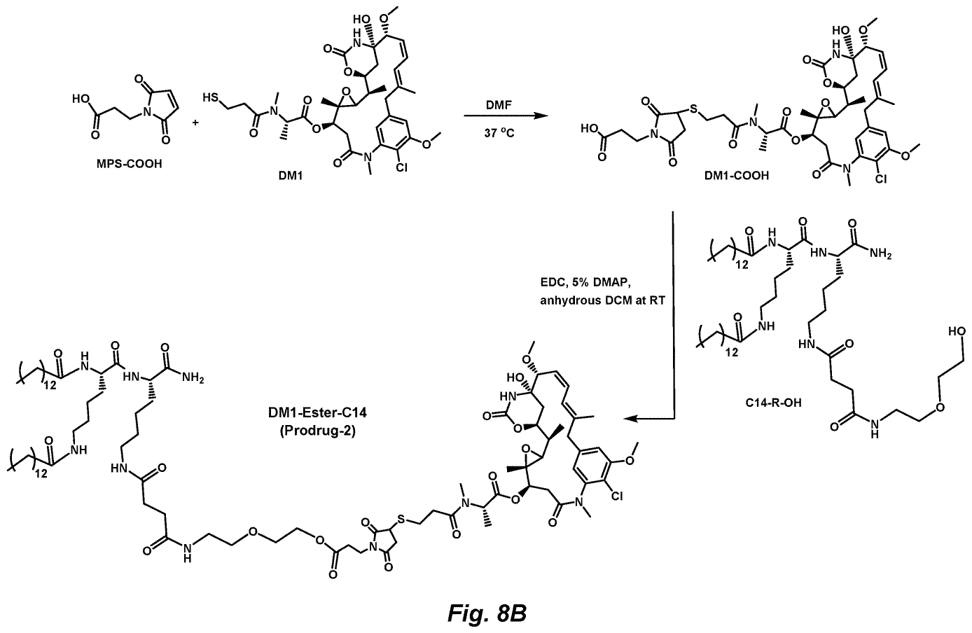

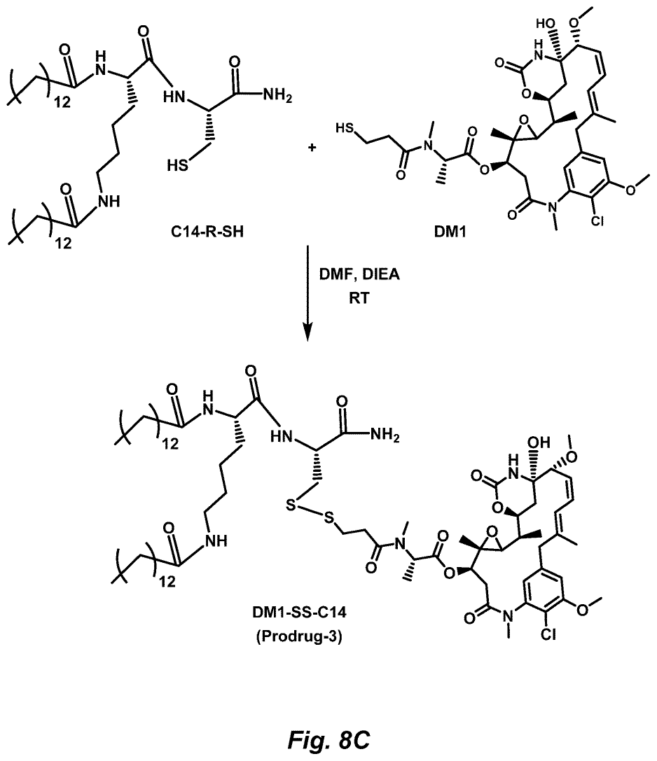

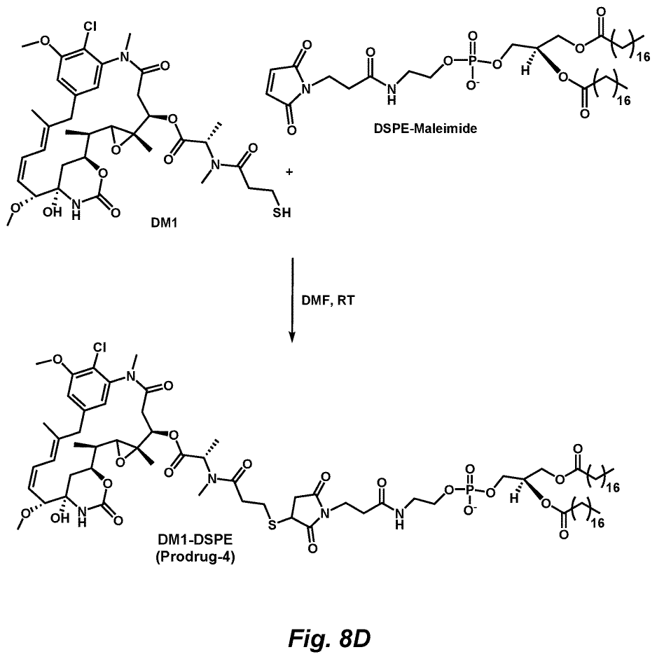

We synthesized four prodrug molecules: DM1-Amide-C14, DM1-Ester-C14, DM1-SS-C₁₄, and DM1-DSPE, using linkage moieties (1), (2), (3), and (4), respectively. To incorporate DM1-Prodrug molecules in liposomal formulations, we used lipid-conjugate versions with different chemical linkage moieties (FIG. 3a). As the anchoring lipid tail, C₁₄ (myristic acid) was selected because it provided optimal solubility in typical solid-phase reagents such as dichloromethane (DCM), chloroform, and dimethylformamide (DMF), during synthesis and particle preparation. The C₁₄-C₁₈ tails enable facile insertion into the bilayer of the liposome without destabilizing the nanoparticles or affecting cellular uptake. The linkage moieties (1), (2), and (3) were synthesized, while linkage moiety (4) was a natural part of DSPE, and the purities of the DM1-Prodrug molecules were confirmed using reverse-phase high-performance liquid chromatography (RP-HPLC) (FIG. 3b,). The purity of prodrug molecules were determined to be >95% via RP-HPLC analytical injections using a Zorbax C3 semi-preparative column. Details for the preparation of DM1-Prodrugs are provided in Example 2 (see FIG. 8 for relevant synthetic schemes) and the molecular masses were confirmed MALDI mass spectrometry analysis as shown in Table 1.

TABLE 1
MALDI mass spectrometry analysis of DM1-Prodrug molecules
to confirm the identity of synthesized DM1-Prodrug conjugates.

| Molecule | Expected Mass (Da) | Observed Mass (Da) |
|---|---|---|
| Prodrug-1 | 1581.9 | 1605.01 |
| (DM1-Amide-C14) | | (with Na adduct) |
| Prodrug-2 | 1769.0 | 1792.11 |
| (DM1-Ester-C14) | | (with Na adduct) |
| Prodrug-3 | 1403.79 | 1426.88 |
| (DM1-SS-C14) | | (with Na adduct) |
| Prodrug-4 | 1634.9 | 1658.96 |
| (DM1-DSPE) | | (with Na adduct) |

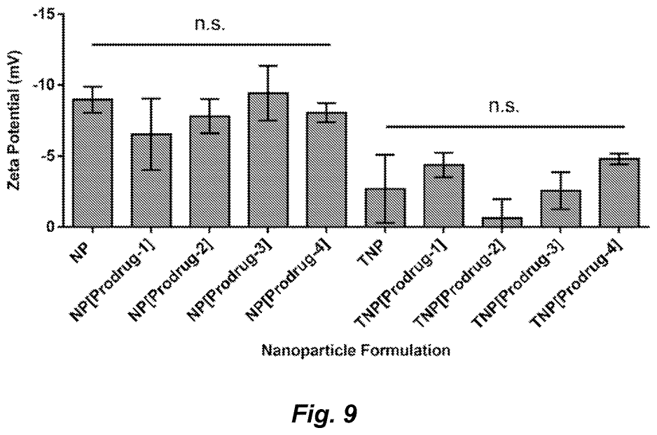

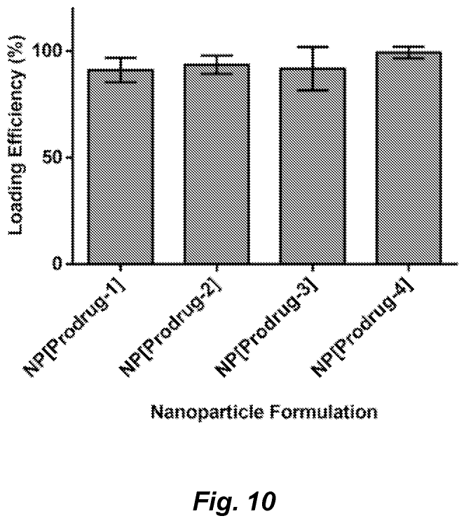

Liposomal prodrug formulations were prepared using specific stoichiometric quantities of DSPC, mPEG-DSPE, Cholesterol, DM1-Prodrug, CD138pep-lipid (FIG. 3c; details in methods section). Both PEGylated lipids and cholesterol were included in the formulation since they provide extended circulation half-life and improve particle stability both in experimental and clinical research. For the formation of DM1-Prodrug incorporated nanoparticles, liposomes were prepared and extruded through a 100 nm polycarbonate membrane to create liposomes (FIG. 3c). The NP[Prodrug] and TNP[Prodrug] formulations yielded similar dynamic light scatter (DLS) and transmission electron microscopy (TEM) results with an average diameter of 100 nm with high reproducibility and stability, showing that the incorporation of the prodrugs along with targeting peptide-lipid conjugate did not alter the size of the nanoparticles (FIG. 3d) Both NP[Prodrug] and TNP[Prodrug] formulations yielded similar dynamic light scatter (DLS) results with an average diameter of 100 nm with higher producibility and stability, emphasizing that the incorporation of the prodrugs along with targeting peptide-lipid conjugate did not alter the size of the nanoparticles. The size of the lipid nanoparticles falls within the particle size range required for the passive targeting of tumors via the EPR effect. The zeta potential of nanoparticles does not change significantly due to incorporation of different DM1-Prodrug (FIG. 9). This is expected due to lack of ionizable components. In contrast, addition of CD138pep-lipid increased the zeta potential of TNP formulations slightly due to the presence of oligolysine content in the conjugate. We were able to incorporate 5 mol % of DM1-Prodrug in the liposomal formulation with >90% encapsulation efficiency (FIG. 10). Our multifaceted synthetic approach allowed precise control over the molar ratio of DM1 presented on the lipid nanoparticle and ensured higher nanoparticle purity and reproducibility.

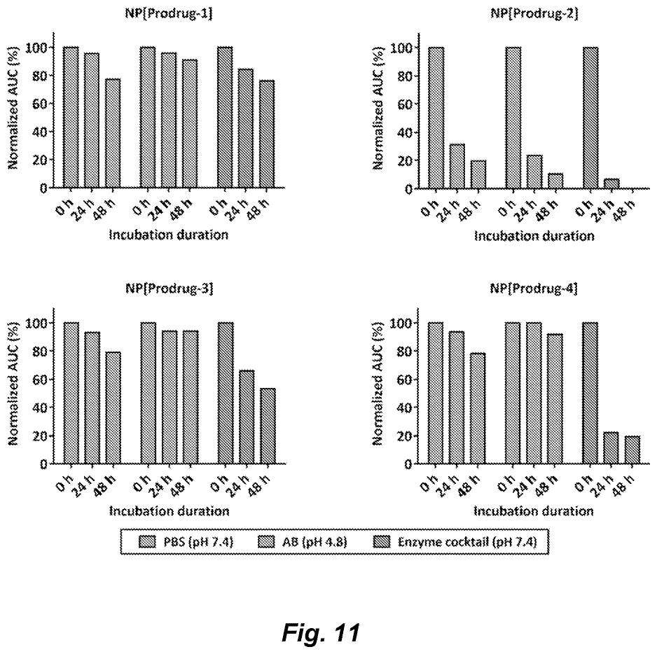

After determining the loading efficiency of DM1-Prodrug, the release of the active DM1 from the liposomes was evaluated using an analytical column in HPLC. We observed that the nanoparticles were able to retain the DM1-Prodrugs at 37° C., at pH 7.4 and 4.8 over the extended period of time (up to 48 h), and no active drug release was observed with the exception of NP[Prodrug-2](FIG. 11). The sustained retention is advantageous in nanoparticle delivery because it prevents leakage of the drug prior to reaching the target site and ensures its delivery in larger quantities to be released at the tumor site. Upon cellular uptake of nanoparticles, active drug release is facilitated by enzymatic degradation of liposomes and linking moiety. Hence, the high retention of DM1-Prodrug in the nanoparticles highlights its potential for improved efficacy in vivo relative to the free drug.

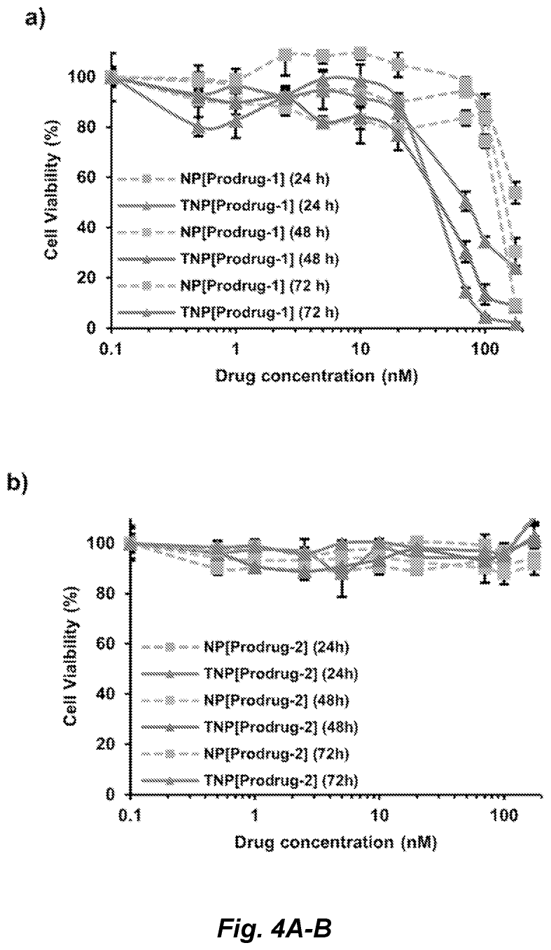

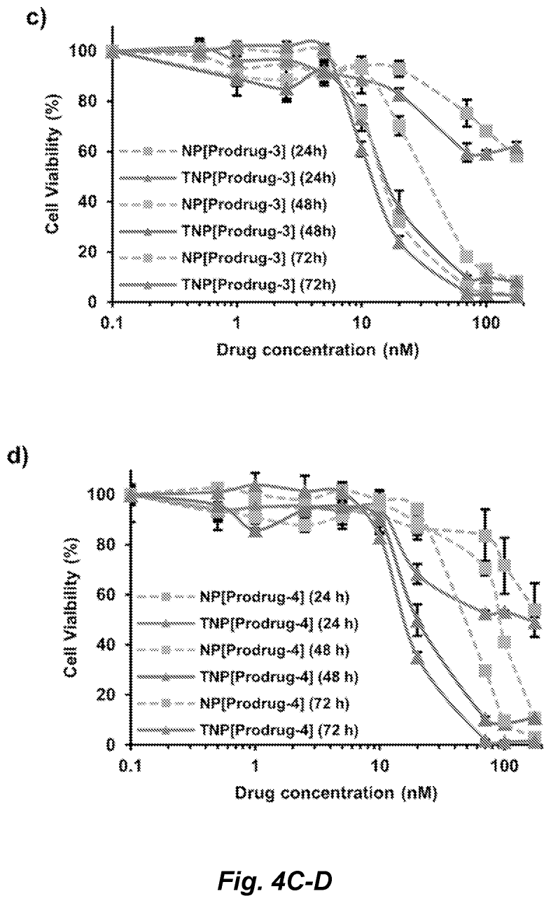

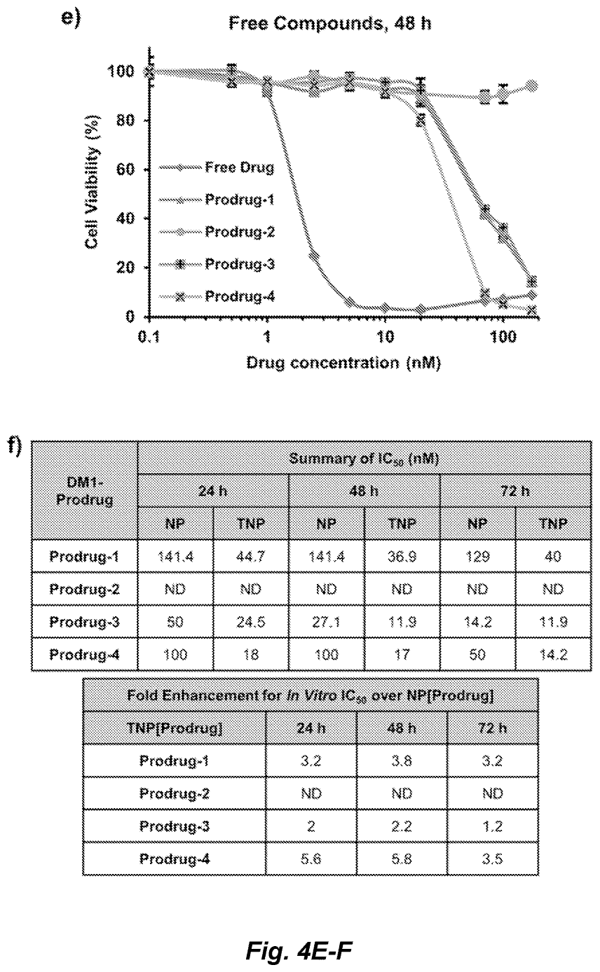

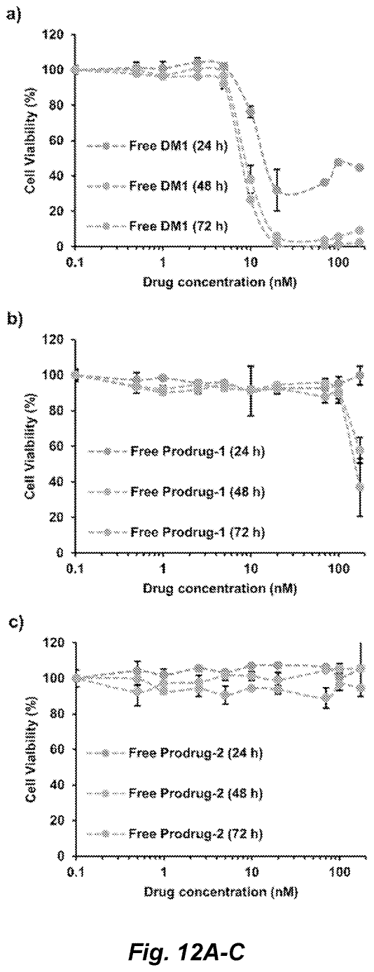

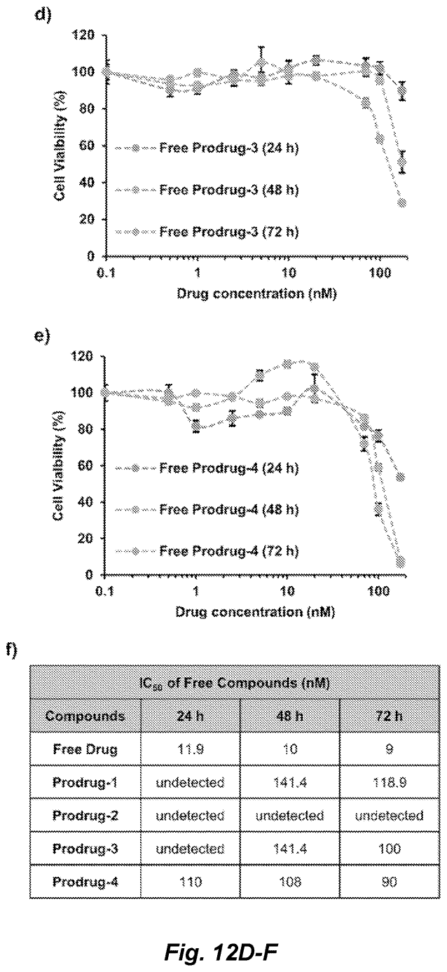

Next, we evaluated the efficacy of liposomal DM1-Prodrug formulations against MM cells in vitro. For this, NCI-H929 cells were incubated with NP[Prodrug], TNP[Prodrug], free DM1-Prodrug or free drug for 24 h, 48 h and 72 h, and cell viability was determined using cell counting kit-8 (CCK-8) reagent. Free DM1 had an IC₅₀ values of 12 and 9 nM with NCI-H929 cell line at 24 and 72 hours respectively (FIG. 12). The liposomal nanoparticles demonstrated a slight reduction in cytotoxic effect upon NCI-H929 cells compared to free DM1 (FIG. 4a-4e). The differences in the IC₅₀ values between free drug and the nanoparticles is possibly attributed to the release kinetics of active DM1 from nanoparticles.

Free drugs enter cells via passive diffusion, while nanoparticles are internalized by endocytosis which adds an additional step and causes a delay in cellular cytotoxicity. We observed TNP[Prodrug-1] and TNP[Prodrug-4] to have IC₅₀ values of 37 and 17 nM at 48 h respectively, which represent maximum enhancements of ~4 and ~6 fold improvements compared to their respective non-targeted versions in ideal results, NP[Prodrug-1] and NP[Prodrug-4](FIG. 4f). On the other hand, TNP[Prodrug-3] had about ~2 fold improved cytotoxic effect compared to the non-targeted version. NP[Prodrug-2] and TNP[Prodrug-2] displayed zero cytotoxicity within tested range. The differences in the IC₅₀ values of various DM1-Prodrug formulations can be attributed to differences in chemistries of the linkers that are needed to be hydrolyzed and the catalytic activity of the relevant catalysts/enzymes required for the release of active drug, such as amidase, esterase, phosphatase and glutathione. Our cytotoxicity results indicate that we were able to tune the cytotoxic effects in vitro using different prodrug formulations of the DM1 (FIG. 4f).

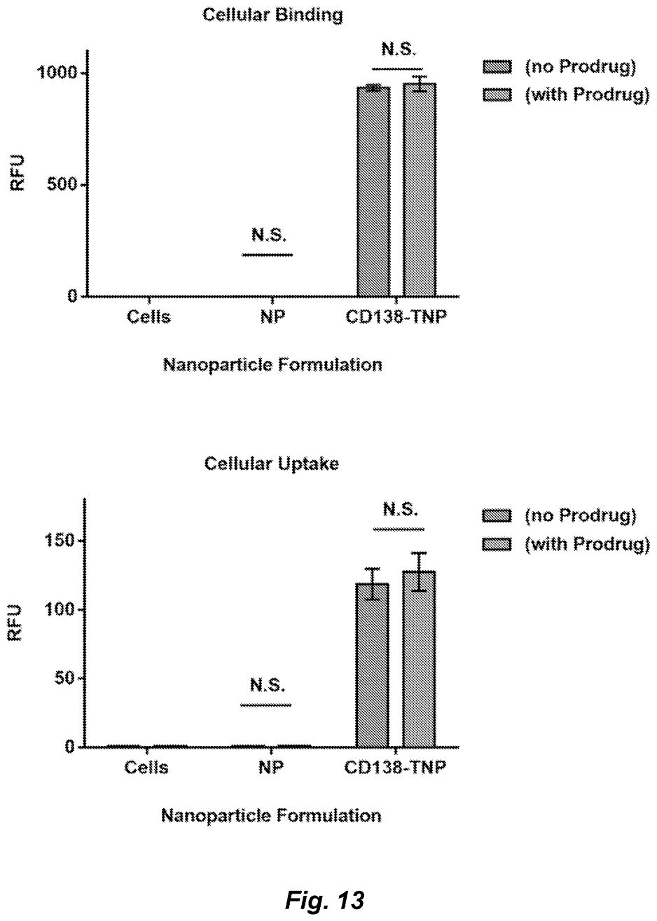

Most importantly, by using an active targeting approach and stable prodrug formulation, such as Prodrug-1 and Prodrug-4, we were able to reduce MM cell viability with higher efficacy. This improved cytotoxic characteristic of Prodrug-4, along with the pronounced targeting of CD138 (FIG. 13), has the potential to deliver immensely potent DM1 safely with enhanced therapeutic index and negligible off-target effects in vivo. In addition to cytotoxic assessment, we evaluated if the liposomal nanoparticles were inducing apoptosis of MM cells by observing early and late apoptotic markers, such as Annexin-V and Propidium Iodide (PI). NCI-H929 cells were incubated with 20 nM equivalence of DM1 concentration either in the form of one of the liposomal prodrug formulations, or the free drug for 24 h. We noticed free DM1 and all TNP[Prodrug] formulations (except for Prodrug-2) were able to induce apoptosis to a similar extent and in an improved manner compared to their respective non-targeted nanoparticle versions (from apoptosis assay, free DM1 and all TNP[Prodrug] formulations (except for Prodrug-2) were observed to induce apoptosis of MM cells).

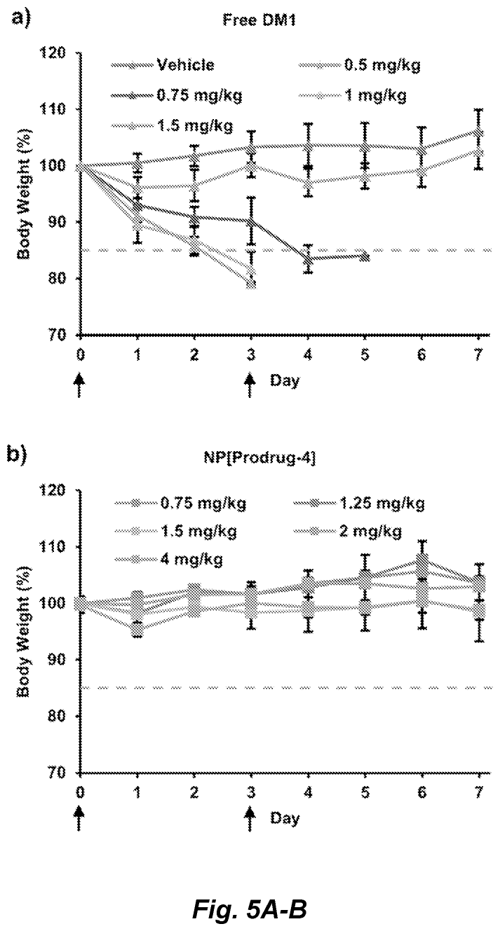

Prior to carrying out the in vivo mouse efficacy study, we determined the maximum tolerated dose for both free DM1 and liposomal Prodrug-4 formulations by evaluating them on healthy NOD-SCID mice. When mice were injected with free DM1 at a dose over 0.5 mg/kg twice on days 0 and 3, we observed that all animals lost over 15% of their body weight and were morbid before the end of the week (FIG. 5). Nevertheless, mice that were treated with liposomal nanoparticle formulation displayed barely noticeable side-effects even after administering two doses of 4 mg/kg of equivalent DM1 treatment. We concluded that liposomal prodrug formulations made treatment with DM1 possible in comparison to free DM1, where its intense systemic toxicity made it a non-viable treatment option. With this approach of liposomal prodrug formulation, even the non-targeted formulations already achieved an improved therapeutic index for DM1.

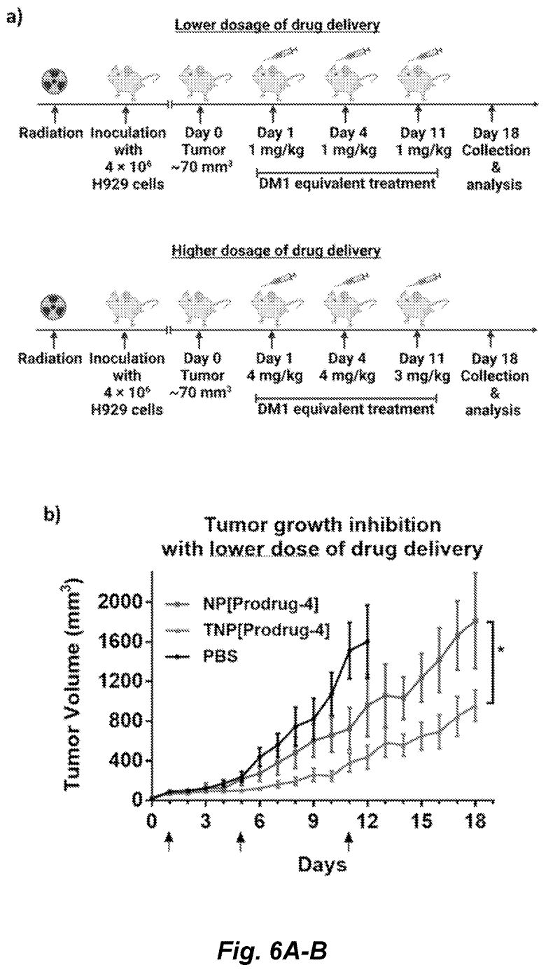

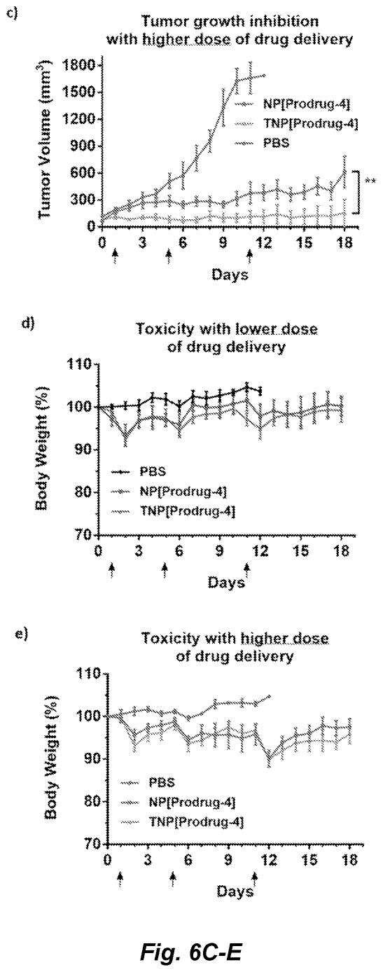

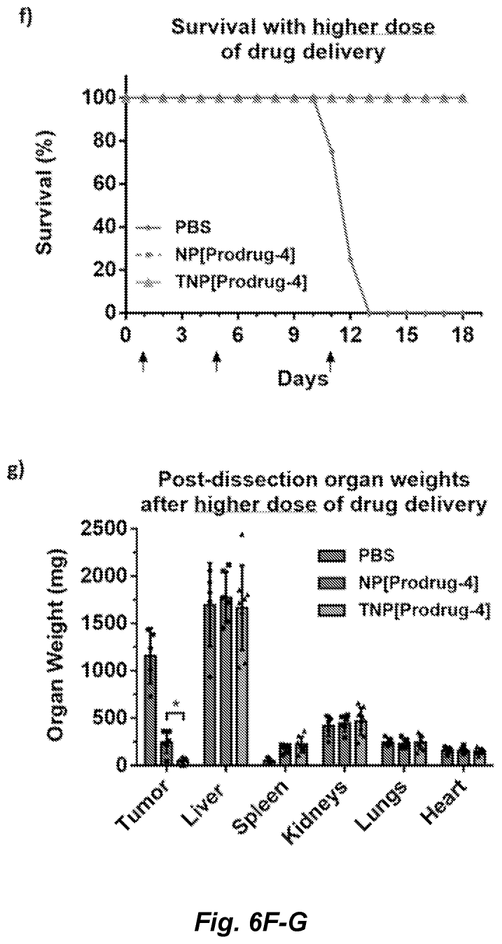

To evaluate the therapeutic potential of liposomal Prodrug-4 formulation, subcutaneous NCI-H929 tumor-bearing NOD-SCID mice were randomized into treatment groups when tumors reached a volume of 70 mm³. Mice were retro-orbitally injected with PBS (control), NP[Prodrug-4], or TNP[Prodrug-4] on indicated days (either 1 mg/kg DM1 equivalent on day 1, 5 and 11, or 4 mg/kg DM1 equivalent on day 1 and day 5, and 3 mg/kg DM1 equivalent on day 11) (FIG. 6a). Mice were monitored for tumor growth inhibition and systemic toxicity. Both NP[Prodrug-4] and TNP[Prodrug-4] demonstrated efficacy in tumor growth inhibition (FIG. 6b, 6c), while the tumor growth in the control group continued in an exponential manner. Some of the mice in the control group were sacrificed before the end of the study period as the tumor burden became over the maximum allowed tumor size in accordance with IACUC protocols (>2000 mm³).

The improved efficacy and negligible systemic toxicity of both NP[Prodrug-4] and TNP[Prodrug-4] can be attributed to the typical combination of EPR effect with controlled drug release at the tumor site. Consistent with in vitro cytotoxicity results, TNP[Prodrug-4] demonstrated a modest but still statistically significant (with a p-value <0.05) enhancement in tumor growth inhibition relative to NP[Prodrug-4] when treated with lower dosage of DM1 equivalent treatments. Most importantly, TNP[Prodrug-4] achieved ~99% tumor growth inhibition compared to control by day 10 and was statistically significant (with a p-value <0.0001) for the higher dosage of DM1 equivalent treatments. Essentially, the controlled release of the active DM1 from TNP[Prodrug-4] played a major role in significant improvement of tumor growth inhibition due to incorporation of active targeting elements (CD138pep) in comparison to non-targeted NP[Prodrug-4] formulation. Importantly, mice showed negligible systemic toxicity with both NP[Prodrug-4] and TNP[Prodrug-4] during the observation period (FIG. 6d, 6e).

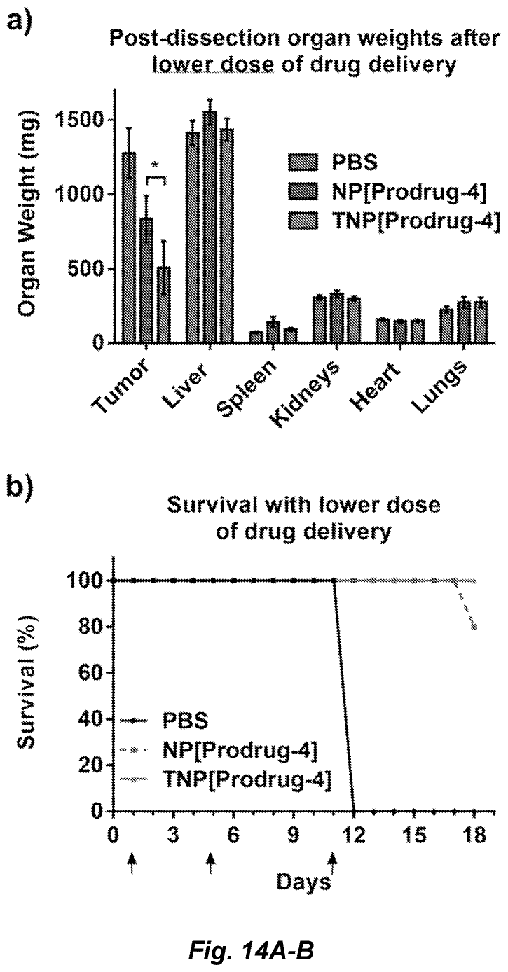

Nevertheless, mice in both NP[Prodrug-4] and TNP[Prodrug-4] treated groups have a survival rate of 100%, while mice in the control group had to be sacrificed early due to large tumor burden (FIG. 6f, FIG. 14). Animals in both groups lost approximately 10% of their initial body weight after each treatment, however, they recovered the lost weight within the next 2-3 days. This observation was supported by the major organ weights post-dissection, where all the nanoparticle formulations displayed similar organ weights to that of the PBS control mice (FIG. 6g, FIG. 14). We also performed H&E staining of the major organs to qualitatively determine whether nanoparticles had toxic effects during in vivo efficacy study. H&E staining of the major organs and tumor showed no toxicity of nanoparticles during in vivo efficacy study. Overall, no noticeable differences in major organs were observed among the treatment groups and control. Importantly, the TNP[Prodrug-4] of higher and lower dosage delivery exhibited more cell death in tumor tissues compared to control and non-targeted versions. This analysis revealed that there were no observable differences in the major organs between the treatment groups and the control (PBS) group from H&E staining. Furthermore, and of significance, the tumors from the higher dosage TNP[Prodrug-4] group showed comparatively less nucleated cells (or more cell death) compared to the tumors from other groups.

Despite examples in literature for DM1's high potential to treat MM and having been FDA approved in the form of an antibody drug-conjugate (ADC) for breast cancer treatment in the last decade, the significant systemic toxicity of DM1 that results in severe side-effects hinder it from being used in treating MM, or other cancers, in the clinic. Therefore, as demonstrated herein, a prodrug formulation incorporated into targeted liposomal nanoparticles can facilitate the safe and efficacious use of the DM1 therapeutic potency in a clinical setting.

An ideal prodrug formulation for effective treatment of cancers should possess the following characteristics: 1) flawless incorporation in nanoparticles, 2) resilience to premature hydrolysis/degradation prior to reaching target cells, and 3) efficient conversion to active form upon reaching the target. In search of the ideal prodrug characteristics, we developed four alternate DM1-Prodrug molecules using functional linker chemistries that include either an amide, an ester, a disulfide, or a phosphodiester. These prodrug linker chemistries were evaluated in a CD138pep-targeted liposomal nanocarrier to identify the DM1-Prodrug that delivered the efficacy in a MM tumor model. Following a two-step approach, we assessed the prodrug candidates. The first step of evaluations started with in vitro experiments of cellular binding, uptake and cytotoxicity for the different formulations prior to moving forward with the second step, in vivo animal testing.

In vitro evaluations provide a decent starting point to identify and eliminate potential negative consequences relating to difficulties in synthetic scale up, inadequate formulation parameters, weak peptide specificity, and poor therapeutic efficacy. Using the relative bond-stability of prodrugs, evaluated in the form of liposomal formulations, we ranked the four DM1-Prodrug molecules according to their susceptibility to hydrolysis under different buffer conditions (FIG. 11). NP[Prodrug-1] exhibited the slowest hydrolysis among the prodrugs under different buffer conditions, meanwhile NP[Prodrug-2] was observed to rapidly degrade under identical conditions. NP[Prodrug-3] and NP[Prodrug-4] had negligible hydrolysis in both low pH (4.8 similar to conditions of TME) and physiological pH (7.4). Nonetheless, the enzyme cocktail, that was used to evaluate the susceptibility of NP[Prodrug] to enzymatic activity, accomplished a more thorough hydrolysis of NP[Prodrug-4] compared to other prodrug molecules, indicative of NP[Prodrug-4] formulation's potential for efficient conversion to active drug.

Following studies of relative chemical bond stabilities, we evaluated potency of the prodrugs by analyzing in vitro cellular cytotoxicity. Despite having a slower hydrolysis profile, the TNP[Prodrug-1] demonstrated ~3.8-fold improvement in IC₅₀ over the non-targeted formulation indicating a significant increase in cellular cytotoxicity (FIG. 4, 11). Interestingly, NP[Prodrug-2] and its targeted version did not display significant cytotoxicity within tested range. Upon hydrolysis of Prodrug-2, the resulting structure of the “active drug” would be identical to the structure of DM1-COOH and is responsible for the shift in IC₅₀ (FIG. 3b).

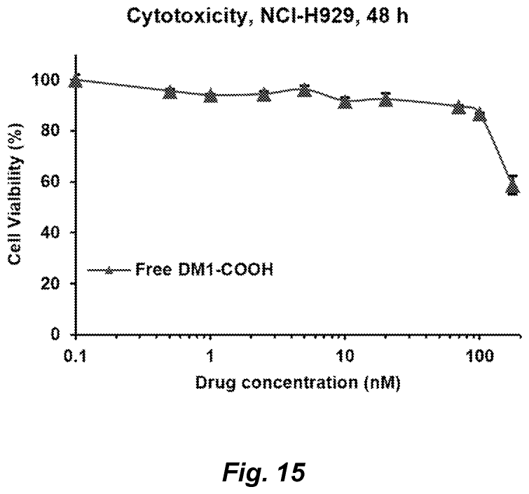

To confirm our prediction, we performed a cytotoxicity assay with the intermediate compound DM1-COOH and observed a barely noticeable reduction in cell viability (FIG. 15). Nevertheless, both NP[Prodrug-2] and TNP[Prodrug-2] were deemed relatively less toxic, since cells were observed to be undergoing early apoptosis too slowly compared to other formulations as indicated by Annexin V staining (From apoptosis assay, free DM1 and all TNP[Prodrug] formulations (except for Prodrug-2) were observed to induce apoptosis of MM cells). On the other hand, slight differences of IC₅₀ were observed for between targeted and non-targeted formulations for Prodrug-3, which indicated that redox sensitive linkage moieties (disulfide bridges) in the prodrug design were not ideal for selective delivery of DM1. We predict that the drug leaking out of the delivery vehicle prior to reaching target tissue would exert cytotoxicity on unintended tissues/organs and leave less drug to be delivered to the tumor resulting in reduced efficacy.

When TNP[Prodrug-4] demonstrated ~6 fold improved IC₅₀ compared to its non-targeted version as well as TNP[Prodrug-1], the winning linker chemistry became clear. Prodrug-4, with a phosphodiester linkage moiety, had the most favorable prodrug characteristics for a nanoparticle formulation. Prodrug-4 was stable on the shelf and in tissue culture conditions prior to cellular uptake along with better IC₅₀, indicating a strong potential for in vivo efficacy. Hence, Prodrug-4 was identified as the lead formulation from in vitro evaluations.

Although the in vitro studies provide certain evidence that help estimate a nanomedicine's potential to treat cancer in animal disease models, they still fall short of providing an accurate picture in predicting the clinical implications due to the increased complexity of the in vivo environment. Factors such as turbulence and shear due to blood flow affecting interactions between peptide ligand and target receptor, off-target binding to healthy cells, phagocytosis and clearance by immune cells and reticuloendothelial system (RES), nanoparticle tumor accumulation via the EPR effect and active transport, tumor penetration, and binding site barrier are present in the in vivo environment. Our established liposomal platform successfully addressed these complications by systematically identifying the impact of each design parameter (such as length of peptide-linker, peptide hydrophilicity, peptide density, nanoparticle size, and PEGylated lipids) in isolation to aid their adjustment to accomplish finely tuned targeted nanoparticles in preclinical mouse studies. Initial TNP formulations with CD138pep targeting to be evaluated in vivo were designed by considering the results we obtained during in vitro evaluations, as well as the results from our previously biodistribution data on various CD138pep loading.

In line with our predictions, MTD study of free DM1 vs NP[Prodrug-4] in healthy animals indicated a much higher dosing of the drug could be safely administered using the NP[Prodrug-4] formulation, which rationalized administration of an 8-fold higher dose than the free drug and deliver a total of 11 mg/kg of equivalent DM1 to achieve efficacy. Our in vivo antitumor efficacy studies established the advantage of TNP[Prodrug-4] compared to NP[Prodrug-4], which we attribute to effective delivery of the drug inside the cancer cells that was achieved by CD138pep targeted nanoparticles. The design of the targeted nanoparticles provides enhanced selectivity towards MM tumor cells as a result of their fine tuning, which achieves higher avidity to MM cells over other lymphocytes.

Both nanoparticle versions exhibited negligible systemic toxicity and reduced tumor burden, while mice in the control group had to be sacrificed earlier due to their prodigious tumor burden. The most significant findings from this study were; 1) prodrug formulations enabled the administration of higher doses of equivalent drug by solving both solubility and systemic toxicity issues, 2) nanoparticle formulation of Prodrug-4 surpassed the free drug by enhancing its safety profile and therapeutic window, and perhaps most importantly, 3) targeted nanoparticles (TNP[Prodrug-4]) outperformed non-targeted particles (NP[Prodrug-4]) by inhibiting tumor growth with a superior efficacy (~99%) in MM tumor bearing mice as a result of improved delivery directly into the cancer cells rather than to their periphery within the tumor tissue.

Combining the unique strength of prodrug strategy and rational design of peptide-targeted liposomal delivery system opens an avenue for developing next generation superior therapeutics, while the personalized medicine approach that can achieve the best patient outcomes is lagging behind. The increased tolerated dose could allow the treatment cycle to be separated over a few more days which would improve the quality of life for the patients during treatment by reducing the frequency and the number of times that they would need to be administered the chemotherapeutic.

While our study focuses solely on the synthesis and application of DM1-Prodrug for liposomal nanoparticle formulations, the strategies described within this disclosure can be implemented in combination with currently clinically approved immunomodulatory drugs or checkpoint inhibitors for better clinical prognosis, disruption of MM cell/bone-marrow interaction, and increased anti-cancer effector functions in relapsed/refractory MM. Further preclinical studies are being performed in our laboratory to evaluate the efficacy of DM1-Prodrug in a variety of cancer models to combat tumor growth in vivo. Novel formulations, such as the targeted liposomal DM1 prodrugs described herein, can reduce the systemic toxicity of approved clinical therapies while maintaining a high anti-tumor efficacy. The formulations described herein can therefore benefit not only MM patients, but also a broader cancer patient population.

### Example 2. Material and Methods

Materials. NovaPEG Rink amide low loading resin, 2-(1H-benzotriazol-1-yl)-1,1,3,3 tetramethyluroniumhexafluorophosphate (HBTU), and all Fmoc-protected amino acids were purchased from EMD Millipore. We purchased N,N-diisopropylethylamine (DIEA), trifluoroacetic acid (TFA), triisopropylsilane (TIS), dimethylformamide (DMF), dichloromethane (DCM), 2-proponol (IPA), acetonitrile (ACN), ethanol, Kaiser test reagents, cholesterol, N,N-diisopropylcarbodiimide (DIC), hydrazine, deoxyribonuclease, trypsin, and chloroform from Sigma-Aldrich (MO, USA). 1,2-ethanedithiol (EDT) was purchased from Alfa Aesar (MA, USA). We obtained 1,2-distearoyl-sn-glycero-3-phosphocholine (sodium salt) (DSPC), and 1,2-distearoyl-sn-glycero-3-phosphoethanolamine-N-[methoxy(polyethyleneglycol)-2000](ammonium salt) (mPEG2000-DSPE) from Avanti Polar Lipids (AL, USA). MPS—COOH was purchased from Quanta Biodesign (OH, USA). DSPE-maleimide was bought from NanoSoft Polymers (NC, USA). DM1 was purchased from MedChemExpress (NJ, USA). The dead cell apoptosis kit, 4-(dimethylamino) pyridine (DMAP), HyClone fetal bovine serum (FBS), 3,3′-Dioctadecyloxacarbocyanine Perchlorate (DiO), myristic acid (99%), Fab digestion buffer, L-Cysteine-HCL, immobilized pepsin, immobilized papain, and phosphate-buffered saline (PBS, powdered, pH 7.4) were purchased from Thermo Fisher Scientific (MA, USA). CCK-8 was purchased from Dojindo Laboratories (Kumamoto, Japan). Carbon films were purchased Electron Microscopy Sciences (PA, USA).

Synthesis and Characterization of Peptide-Lipid Conjugates. Peptide lipid conjugates and linkage molecules were synthesized using standard Fmoc-based solid phase peptide synthesis method as described in Stefanick et al., ACS Nano. 7 (2013) 2935-2947. www.doi.org/10.1021/nn305663e; Omstead et al., J. Hematol. Oncol. 13 (2020) 145. www.doi.org/10.1186/s13045-020-00965-4. Synthesized peptide-lipid conjugates were cleaved from resin and purified using a Zorbax C3 semi-preparative column and Agilent 1200 reversed-phase high-performance liquid chromatography (RP-HPLC) with a gradient of 60-80% for 10 min in a two-phase system of IPA/ACN/H2O mixture and H2O. Final DM1-Prodrug molecules were generated by reacting the linkage moieties with DM1 in DMF at room temperature overnight. After removing the reaction solvent, the prodrugs were purified via RP-HPLC. The products were characterized by MALDI ultraflex, and their purities (>95%) were determined by the RP-HPLC analytical injections using a Zorbax C3 semi-preparative column.

Liposomal Nanoparticle Preparation. The liposomal nanoparticles were prepared by dry film hydration and extrusion. Briefly, lipids were mixed in chloroform, at specific stoichiometry by applying the formula (95-x):5:5:5:x which indicated ratios of DSPC:mPEG-DSPE:Cholesterol:DM1-Prodrug:CD138pep-lipid, where x denotes the molar ratio of CD138pep-lipid conjugate present on the surface of the nanoparticle. CD138pep-lipid was used at 1 mol % and 0.1 mol % in the nanoparticle formulations for in vitro optimization and in vivo efficacy studies respectively, based on the previous report [21]. Later, lipid mixtures were dried to form a thin film using nitrogen gas, and then placed under vacuum overnight to remove residual solvent. The lipid films were hydrated with PBS (pH 7.4) at 65° C. for 7 min by gentle agitation and extruded at 65° C. through a polycarbonate membrane using Avanti Polar Lipid extruder set.

Liposomal Nanoparticle Sizing. Dynamic light scattering (DLS) analysis was performed using NanoBrook Omni Particle Size Analyzer (Brookhaven Instruments Corp.), as described in Kiziltepe et al., Blood Cancer J. 2 (2012). www.doi.org/10.1038/bcj.2012.10.

Imaging of Liposomal Nanoparticles. Transmission electron microscopy (TEM) was performed to image the 100 nm nanoparticles by using an JEOL TEM 2011. Briefly, nanoparticles were prepared at 500 uM of lipid concentration incorporating DM1-Prodrug. Imaging samples were prepared by following the negative staining method. Briefly, 5 μL of nanoparticle suspensions were placed on a clean parafilm, followed by placing a plasma cleaned grid on the sample drop for 10 seconds and then blotting the grid on the side to drain excess using filter paper. This step was repeated 5 times consecutively. Finally, the grid was placed on a UranyLess solution drop for 40 seconds, then the grid was blotted with filter paper to drain excess. Grids were stored overnight at room temperature for imaging next day. Images were acquired at multiple scales to assess the overall distribution of the specimen. The transmission electron microscopy (TEM) images of all nanoparticle formulations showed similar nanoparticle morphology and polydispersity with an average diameter of 100 nm.

Zeta potential Measurement. Zeta potential was measured using the Smoluchowski approximation on a NanoBrook Omni (Brookhaven Instruments Corp.). Samples were diluted to a lipid concentration of 125 μM and analyzed for 30 cycles per run.

Drug Loading Efficiency. The loading efficiency of DM1-Prodrug incorporated within the liposomes was determined by loading 5 mol % DM1-Prodrug of the total lipid constituents. Accordingly, the molar percentage of DSPC decreased with respect to the drug loading, while we kept the molar percentage of mPEG2000 and cholesterol constant at 5 mol %. Nanoparticles were prepared and purified via the liposome extruder purification (LEP) method. Then, DM1-Prodrug in the nanoparticle formulations was measured via RP-HPLC at 220 nm and 280 nm on an Agilent 1200 series system.

Stability and Release Analysis of DM1. Samples were prepared at 25 mM of lipid concentration incorporating DM1-Prodrug. Nanoparticles were prepared using dry film hydration and extrusion as mentioned earlier. Then, samples were incubated in phosphate buffer (PBS, pH 7.4), acetate buffer (AB, pH 4.8) and enzyme cocktail (pH 7.4) at 37° C. with gentle shaking. Enzyme cocktail was prepared in 600 μL digestion buffer using 20 mg of deoxyribonuclease, 30 mg of trypsin and 60 mg of Cysteine.HCl. Later, 20 μL of prepared nanoparticles were incubated in 45 μL of enzyme cocktail, followed by adding 15 μL of pepsin, 15 μL of papain, and 50 μL of PBS (pH 7.4). Finally, samples were collected at t=0, 24, and 48 hours for DM1-Prodrug content characterization via RP-HPLC on an Agilent 1200 series system with a semi-preparative Zorbax C₃ column with isopropanol gradients.

Cell Culture. NCI-H929 cells were purchased from ATCC (Rockville, MD). Cells were cultured in RPMI 1640 media (Corning, NY). The cell line was supplemented with 20% fetal bovine serum (FBS), 2 mM L-Glutamine (Gibco, CA), 100U/mL penicillin (Gibco, CA), 100 μg/mL streptomycin (Gibco) and 55 μM 2-mercaptoethanol.

Cellular Binding Assay. 1×10⁵ cells/well were plated in a 24-well plate overnight and incubated at 37° C. incubator. Cells were incubated on ice, for 1 h, with DiO-labeled liposomal nanoparticles in blocking buffer (1.0% bovine serum albumin in PBS). Cells were washed twice with PBS and analyzed with a Guava EasyCyte flow cytometer.

Cellular Uptake Assay. 1×10⁵ cells/well were plated in a 24-well plate overnight and incubated at 37° C. incubator. Liposomal nanoparticles (45 μM total phospholipid concentration) were added to the cells and incubated for 4 h at 37° C. Each nanoparticle formulation contained 0.4 mol % DiO for quantification of cellular uptake. After completing incubation, the cells were washed twice with PBS buffer (pH 7.4) and then trypsinized for 5 min to remove cellular surface-associated nanoparticles. Then, the cells were collected and washed twice with PBS buffer and analyzed by Guava EasyCyte flow cytometer.

Cytotoxicity Assays. 15×10³ cells/well were plated 24 h prior to each experiment in a 96-well plate. The following day, cells were treated with respective cytotoxic agents at varying concentrations. Cytotoxicity was assessed at 24 h, 48 h and 72 h using Cell Counting Kit-8 Reagent via reading absorbance at 450 nm. Viability was normalized to wells containing untreated cells.

Apoptosis Assay. NCI-H929 cells were cultured in the presence of 20 nM of DM1 equivalent concentrations of NP[Prodrug], TNP[Prodrug], and free drug for 24 h. Cells were washed and stained using the Dead Cell Apoptosis Kit by following and optimizing the manufacturer's protocol. Briefly, cells were washed with cold PBS, resuspended in 1X binding buffer, followed by Annexin-V and PI staining for 30 min at room temperature. Staining was performed by adding 7 μL of Alexa Fluor® 488 Annexin-V and 8 μL of 100 μg/mL PI working solution to each 100 μL of cell suspensions. Cells in treatment groups and untreated control were stained with Annexin-V and PI. Fluorescent microscopy images were acquired by using an EVOS® FL Auto Imaging System.

Maximum Tolerated Dose (MTD) Study. Healthy NOD-SCID mice were distributed into treatment groups of 4-5 mice and were treated intravenously via retro-orbital injections with NP[Prodrug-4], or free drug at various concentrations of equivalent DM1. Free drug (DM1) was administered according to the recommendations of the manufacturer. Briefly, Free drug treatments were prepared by dissolving in 10% DMSO and 90% saline containing (20% Hydroxypropyl-β-cyclodextrin as excipient) and then administered intravenously into mice.

MM Xenograft Mouse Model. NOD-SCID mice (acquired from Littlepage Lab at Harper Cancer Institute, Notre Dame, IN) were irradiated with 150 rad and were inoculated subcutaneously with 4×10⁶ NCI-H929 cells. When tumors reached a volume of 70 mm³, mice were distributed into treatment groups of 5-6 mice and were treated intravenously via retro-orbital injections with NP[Prodrug-4], TNP[Prodrug-4], or PBS. For lower dose of drug delivery, mice were injected with 1 mg/kg DM1 equivalent on days 1, 5 and 11. For higher dose of drug delivery, mice were administered with 4 mg/kg DM1 equivalent on day 1 and day 5, and 3 mg/kg DM1 equivalent on day 11. Animals were monitored for body weight and tumor volume. Tumor volume was measured via calipers (volume=0.5×length×(width)²). Tumor inhibition was calculated by comparing the average tumor volume of TNP[Prodrug-4] and Control at Day 18, while subtracting their average tumor volumes at Day 0. Mice were treated humanely and in accordance with the protocol approved by the Institutional Animal Care and Use Committee (IACUC) at the Freimann Life Science Center (Notre Dame, IN).

Histology Imaging. Tissues were fixed using 10% neutral buffered formalin and processed for hematoxylin and eosin staining at the Histology Core Facility, NDIIF, University of Notre Dame. Imaging was performed using a Keyence BZ-X810 brightfield microscope equipped with a 4× (N.A. 0.13) and 40× (N.A. 0.95) objective lenses.

### Example 3. Pharmaceutical Dosage Forms

The following formulations illustrate representative pharmaceutical dosage forms that may be used for the therapeutic or prophylactic administration of a composition described herein, or a composition specifically disclosed herein comprising the disclosed nanoparticles (hereinafter referred to as ‘Composition X’):

| (i) Tablet 1 | mg/tablet |
|---|---|
| ‘Composition X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| ii) Tablet 2 | mg/tablet |
|---|---|
| ‘Composition X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Composition X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| ‘Composition X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/ml |
|---|---|
| ‘Composition X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ‘Composition X’ | 20 |
| Oleic acid | 10 |
| Trichloromonofluoromethane | 5,000 |
| Dichlorodifluoromethane | 10,000 |
| Dichlorotetrafluoroethane | 5,000 |

| (vii) Topical Gel 1 | wt. % |
|---|---|
| ‘Composition X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |

| (viii) Topical Gel 2 | wt. % |
|---|---|
| ‘Composition X’ | 5% |
| Methylcellulose | 2% |
| Methyl paraben | 0.2% |
| Propyl paraben | 0.02% |
| Purified water | q.s. to 100 g |

| (ix) Topical Ointment | wt. % |
|---|---|
| ‘Composition X’ | 5% |
| Propylene glycol | 1% |
| Anhydrous ointment base | 40% |
| Polysorbate 80 | 2% |
| Methyl paraben | 0.2% |
| Purified water | q.s. to 100 g |

| (x) Topical Cream 1 | wt. % |
|---|---|
| ‘Composition X’ | 5% |
| White bees wax | 10% |
| Liquid paraffin | 30% |
| Benzyl alcohol | 5% |
| Purified water | q.s. to 100 g |

| (xi) Topical Cream 2 | wt. % |
|---|---|
| ‘Composition X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference, and in particular, Khan et al., Biomaterials 292 (2023), 121913 (www.doi.org/10.1016/j.biomaterials.2022.121913). No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A nanoparticle comprising:
a prodrug comprising a drug-lipid conjugate, wherein a chemical linker group is positioned between a drug and a lipid of the drug-lipid conjugate;
a targeting moiety-lipid conjugate;
a polyethylene glycol-lipid conjugate;
cholesterol; and
a bulk lipid.

2. The nanoparticle of claim 1, wherein the drug comprises mertansine (DM1): 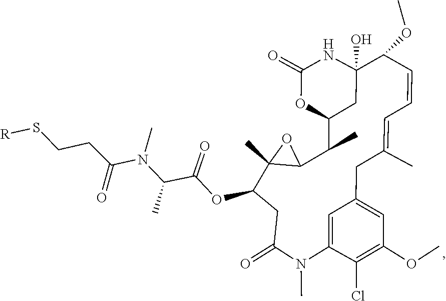
wherein R is the chemical linker group that links the drug to the lipid of the drug-lipid conjugate.

3. The nanoparticle of claim 1, wherein the chemical linker group comprises an amide linker, an ester linker, a disulfide linker, or a phosphodiester linker.

4. The nanoparticle of claim 3, wherein
a) the lipid of the drug-lipid conjugate and the amide linker comprise moiety (1): 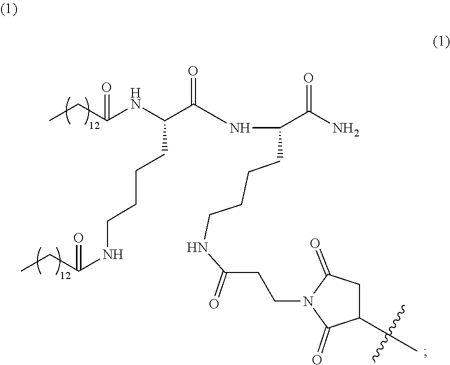
b) the lipid of the drug-lipid conjugate and the ester linker comprise moiety (2): 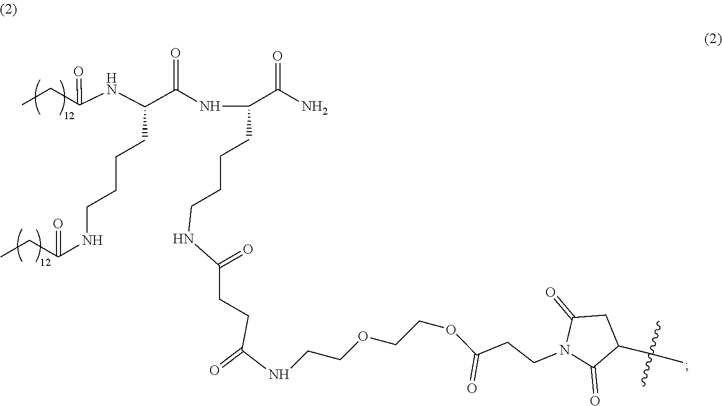
c) the lipid of the drug-lipid conjugate and the disulfide linker comprise moiety (3): 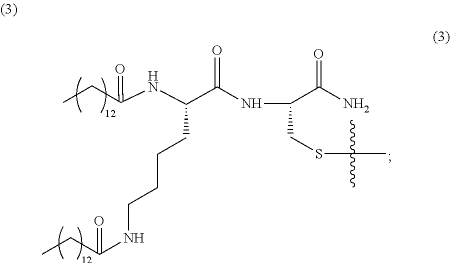
d) the lipid of the drug-lipid conjugate and the phosphodiester linker comprise moiety (4):

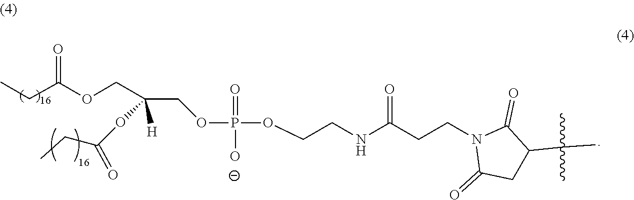

5. The nanoparticle of claim 1, wherein the prodrug comprises:

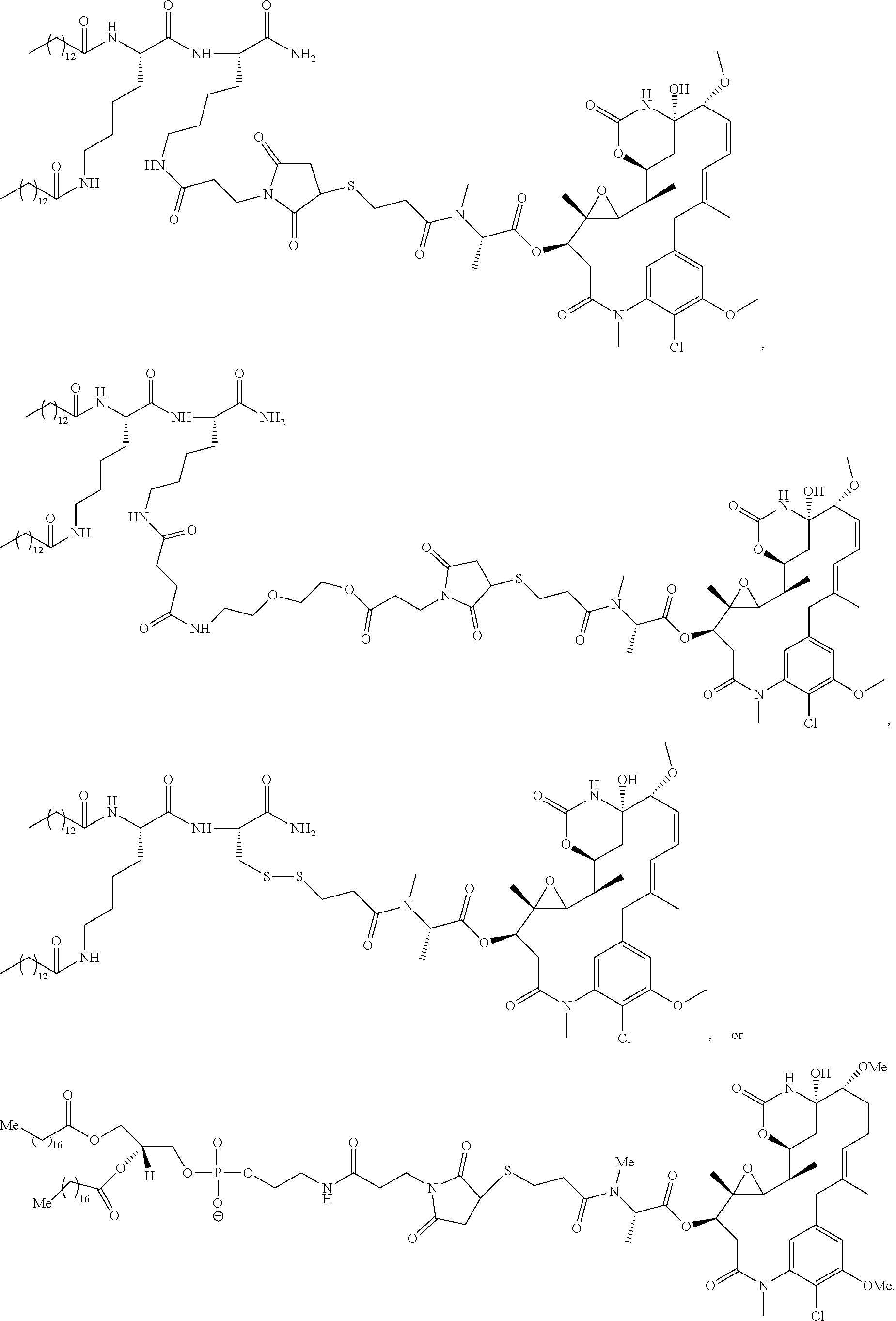

6. The nanoparticle of claim 1, wherein a lipid of the targeting moiety-lipid conjugate and a lipid of the prodrug-lipid conjugate comprise a C₁₄-C₁₈ fatty acid.

7. The nanoparticle of claim 6, wherein the C₁₄-C₁₈ fatty acid is a myristic acid moiety, a palmitic acid moiety, or a stearic acid moiety.

8. The nanoparticle of claim 1, wherein the bulk lipid comprises 1,2-distearoyl-sn-glycero-3-phosphocholine (DSPC).

9. The nanoparticle of claim 1, wherein a targeting moiety of the targeting moiety-lipid conjugate specifically binds to a CD138 receptor, wherein the targeting moiety comprises an antibody, an antibody fragment, or a peptide.

10. The nanoparticle of claim 9, wherein the peptide comprises an amino acid sequence of RKRLQVQLSIRT (SEQ ID NO: 1).

11. The nanoparticle of claim 1, wherein the targeting moiety-lipid conjugate comprises about 0.01 mol % to about 1 mol % of the nanoparticle by molar composition.

12. The nanoparticle of claim 1, wherein the prodrug-lipid conjugate comprises about 1 mol % to about 10 mol % of a total amount of lipid of the nanoparticle.

13. The nanoparticle of claim 12, wherein the cholesterol comprises about 1 mol % to about 10 mol % of a total amount of lipid of the nanoparticle.

14. The nanoparticle of claim 1, wherein the polyethylene glycol-lipid conjugate comprises 1,2-distearoyl-sn-glycero-3-phosphoethanolamine-N-[methoxy(polyethylene glycol)-2000](ammonium salt) (mPEG2000-DSPE).

15. The nanoparticle of claim 1, wherein the targeting moiety-lipid conjugate further comprises one or more of an ethylene glycol spacer, an oligolysine, and an ethylene glycol-peptide linker disposed between a targeting moiety and a lipid of the targeting moiety-lipid conjugate.

16. A nanoparticle comprising:
a bulk lipid;
a polyethylene glycol-lipid conjugate;
cholesterol;
a prodrug comprising a drug-lipid conjugate, wherein a drug of the drug-lipid conjugate comprises Mertansine (DM1), and a lipid of the drug-lipid conjugate comprises a C₁₄-C₁₈ fatty acid, wherein the DM1 is conjugated to the lipid of the drug-lipid conjugate via a phosphodiester linker moiety; and
a targeting peptide-lipid conjugate comprising a peptide having an amino acid sequence of RKRLQVQLSIRT (SEQ ID NO: 1).

17. The nanoparticle of claim 16, wherein the prodrug comprises

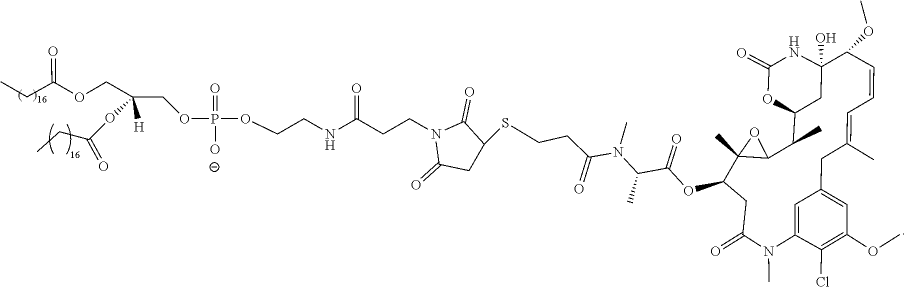

18. A composition comprising the nanoparticle of claim 1 and a pharmaceutically acceptable carrier, excipient, or diluent.

19. A method of treating a subject having multiple myeloma comprising administering to the subject an effective amount of the nanoparticle of claim 1, wherein the nanoparticle treats the multiple myeloma.
